(19) United States
(12) Patent Application Publication
(10) Pub. No.: US 2007/0299013 A1
(43) Pub. Date: Dec. 27, 2007

(54) Exosite-Directed Thrombin Inhibitors

(76) Inventor: Michael Kalafatis, Shaker Heights, OH (US)

Correspondence Address: Mark E Bandy;Fay Sharpe Fagan Minnich & Mckee LLP, 1100 Superior Avenue, Seventh Floor, Cleveland, OH, 44114-2579, US

(21) Appl. No.: 10/571,989

(22) PCT Filed: Jul. 1, 2004
(86) PCT No.: PCT/US04/21487

§ 371 Date: Mar. 13, 2006

(51) Int. Cl. A61K 38/08; A61K 38/07; A61P 7/02; C07K 5/10; C07K 7/06

(52) U.S. Cl. 514/17; 514/18; 530/330

(57) ABSTRACT

Disclosed are an amino acid sequence of the human blood clotting factor Va, peptides containing such sequence, and additional peptides of interest that significantly inhibit thrombin generation. Also disclosed are pharmaceutical compositions containing these peptides and related therapeutic methods for inhibiting thrombin generation and treating blood coagulation disorders.

### CROSS-REFERENCE TO RELATED APPLICATIONS

Applicant claims priority of U.S. Provisional Application No. 60/502,186 filed on Sep. 12, 2003.

This work was supported at least in part by the American Heart Association, a grant (grant HL34575) from the National Institute of Health (NIH), and others. Accordingly, the U.S. government may have certain rights herein.

Reference is made to a “Sequence Listing,” appendix submitted on diskette herewith. The material contained on the diskette is hereby incorporated by reference.

### BACKGROUND

The present discovery relates to the prevention and treatment of blood coagulation disorders. It finds particular application in conjunction with thrombin inhibitors, and will be described with particular reference thereto. However, it is to be appreciated that the present discovery is also amenable to other like applications.

Blood coagulation is a process whereby blood thickness and gradually becomes a clot. The process is vitally important to the stoppage of bleeding when blood vessels are damaged. Blood coagulation occurs through a complex series of molecular reactions, ultimately resulting in conversion of soluble fibrinogen molecules, present in the blood, into insoluble threads of fibrin. The result is a blood clot which consists of a plug of platelets enmeshed in the insoluble fibrin network.

There exist human disorders, called “thromboses,” in which blood clots when it normally should not. Thrombosis is a major cause of death due to occlusion of arteries, which leads to heart attacks, strokes and peripheral ischemia (i.e., local deficiencies in blood supply). Thrombosis can also cause occlusion of venous blood vessels and result in pulmonary emboli.

In order to prevent or treat such thrombotic disorders, therapeutic methods to inhibit clot formation or to dissolve clots have been developed. Existing anticoagulants (that inhibit blood clot formation), for example, include heparin, which greatly increases activity of the physiologic anticoagulant, ATIII, in the blood. Warfarins are anticoagulants that are vitamin K antagonists. Since vitamin K is required for synthesis or functioning of a number of clotting factors (i.e., factors II, VII, IX and X, as well as a-thrombin and proteins C and S), sequestration of vitamin K inhibits coagulation.

The existing blood anticoagulants, however, produce side effects. For example, heparin administration can cause bleeding and thrombocytopenia (i.e., decrease in platelets). A disadvantage of warfarins is that it takes several days for their maximum effect to be realized. As with heparin, bleeding can also be a complication. Warfarins are also teratogens and can cross the placenta, causing fetal abnormalities when administered to pregnant women.

Thrombolytic agents, which dissolve existing clots, are also used therapeutically. Their activity is based on enhancing the generation of plasmin from its plasminogen precursor. Such agents include recombinant TPA and streptokinase. Disadvantages of these thrombolytics include a systemic fibrinolytic activity that can result in bleeding throughout the body. Some thrombolytics (i.e., streptokinase) are also highly antigenic and can cause allergic reactions.

Therefore, there are problematic side effects with existing anticoagulant and thrombolytic drugs. Thus, there exists a need for an improved anticoagulant agent and/or pharmaceutical composition that inhibits coagulation to a greater degree and at a faster rate as compared to currently known agents or compositions, and particular, without the noted unwanted side effects. And, related to this, there exists a need for improved therapeutic methods for treating blood coagulation disorders, e.g. thromboses, by the administration of such improved anticoagulants or pharmaceutical compositions.

### BRIEF DESCRIPTION

In accordance with one aspect of the present discovery, a peptide is provided that comprises a sequence of amino acids which is identical to a sequence of consecutive amino acids found within amino acids 695 to 698 (SEQ ID NO. 10) of the human blood clotting factor Va.

In accordance with another aspect of the present discovery, a peptide is provided which comprises a sequence of amino acids which is identical to a sequence of consecutive amino acids found within amino acids 695 to 6990 (SEQ ID NO. 11) of the human blood clotting factor Va.

In accordance with yet another aspect of the present discovery, a peptide is provided which is adapted to inhibit blood coagulation by inhibiting thrombin generation. The peptide comprises an amino acid sequence DYDY wherein one of the Y amino acids is sulfonated (SEQ ID NO. 12, 13).

In accordance with another aspect of the present discovery, a peptide is provided which is adapted to inhibit blood coagulation by inhibiting thrombin generation. The peptide comprises an amino acid sequence DYDY wherein both of the Y amino acids are sulfonated (SEQ ID NO. 14).

In accordance with a further aspect of the present discovery, a peptide is provided which is adapted to inhibit blood coagulation by inhibiting thrombin generation. The peptide comprises an amino acid sequence DYDYQ wherein one of the Y amino acids is sulfonated (SEQ ID NO. 7, 8).

In accordance with another aspect of the present discovery, a peptide is provided which is adapted to inhibit blood coagulation by inhibiting thrombin generation. The peptide comprises an amino acid sequence DYDYQ wherein both of the Y amino acids are sulfonated (SEQ ID NO. 9).

In accordance with yet another aspect of the present discovery, a pharmaceutical composition is provided which is adapted for inhibiting thrombin generation. The composition comprises a peptide including an amino acid sequence DYDY (SEQ ID NO. 10).

In accordance with yet another aspect of the present discovery, a pharmaceutical composition is provided which is adapted for inhibiting thrombin generation. The composition comprises a peptide including an amino acid sequence DYDYQ (SEQ ID NO. 11).

In accordance with a further aspect of the present discovery, a pharmaceutical composition adapted for inhibiting thrombin generation in a human is provided. The composition comprises a peptide including an amino acid sequence DYDY in which one of the Y amino acids is sulfonated (SEQ ID NO. 12, 13).

In accordance with yet another aspect of the present discovery, a pharmaceutical composition is provided which is adapted for inhibiting thrombin generation in a human. The composition comprises a peptide including an amino acid sequence DYDY in which both of the Y amino acids are sulfonated (SEQ ID NO. 14).

In accordance with a further aspect of the present discovery, a pharmaceutical composition is provided which is adapted for inhibiting thrombin generation in a human. The composition comprises a peptide including an amino acid sequence DYDYQ in which one of the Y amino acids is sulfonated (SEQ ID NO. 7, 8).

In accordance with yet another aspect of the present discovery, a pharmaceutical composition is provided which is adapted for inhibiting thrombin generation in a human. The composition comprises a peptide including an amino acid sequence DYDYQ in which both of the Y amino acids are sulfonated (SEQ ID NO. 9).

In accordance with another aspect of the present discovery, a method for inhibiting thrombin generation in a human patient suffering from a blood coagulation disorder is provided. The method comprises administering to the patient an effective amount of a peptide that includes a sequence of consecutive amino acids found within amino acids 695 to 698 (SEQ ID NO. 10) of the human blood clotting factor Va.

In accordance with yet another aspect of the present discovery, a method for inhibiting thrombin generation in a human patient suffering from a blood coagulation disorder is provided. The method comprises administering to the patient an effective amount of a peptide that includes a sequence of consecutive amino acids found within amino acids 695 to 699 (SEQ ID NO. 11) of the human blood clotting factor Va.

In accordance with another aspect of the present discovery, a method for inhibiting thrombin generation in a patient suffering from a blood coagulation disorder is provided. The method comprises administering to the patient an effective amount of a peptide that includes an amino acid sequence DYDY (SEQ ID NO. 10).

In accordance with another aspect of the present discovery, a method for inhibiting thrombin generation in a patient suffering from a blood coagulation disorder is provided. The method comprises administering to the patient an effective amount of a peptide that includes an amino acid sequence DYDYQ (SEQ ID NO. 11).

### BRIEF DESCRIPTION OF THE DRAWINGS

The present discovery may take form in various components and arrangements of components, and in various techniques, methods, or procedures and arrangements of steps. The referenced drawings are only for purposes of illustrating exemplary embodiments, they are not necessarily to scale, and are not to be construed as limiting the present discovery.

FIG. 1 is a diagram of amino acid sequences of peptides contained in the 680 to 709 region (SEQ ID NO. 1-6) of human blood coagulation factor Va.

FIG. 2A is a graph illustrating the inhibitory effect upon cofactor activity by various peptides (SEQ ID NO. 2-6 AND 21-22) at a concentration of 100 μM.

FIG. 2B is a graph illustrating cofactor activity by two peptides of interest designated herein as HC3 (SEQ ID NO. 4) and HC4 (SEQ ID NO. 5) and a control peptide.

FIG. 2C is a graph illustrating the inhibitory effect of increasing concentration of a peptide of interest HC4 (SEQ ID NO. 5) upon the reaction kinetics of prothrombinase.

FIG. 3A and its inset panel are graphs illustrating cofactor activity by a pentapeptide containing a particular amino acid sequence (SEQ ID NO. 11) in accordance with the present discovery.

FIG. 3B is a graph illustrating the effect of increasing concentration of the pentapeptide containing the amino acid sequence of interest (SEQ ID NO. 11) upon the concentration of prothrombin.

FIGS. 4A and 4B show the effect upon the activation of factor V by reacting thrombin with the pentapeptide of interest (SEQ ID NO. 11) as analyzed by SDS-PAGE (A—activation of factor V by thrombin alone, B—activation of factor V by thrombin reacted with certain peptides).

FIG. 5 and FIGS. 5A-5C illustrate the results of chromatographic trials in which the interaction of peptides with active-site-immobilized thrombin were studied.

FIG. 6A illustrates thrombin generation by certain recombinant molecules.

FIG. 6B illustrates thrombin generation by certain recombinant molecules activated with a certain factor.

FIG. 6C illustrates thrombin generation by certain recombinant molecules activated with a different factor.

FIG. 7 illustrates a kinetic model for the inhibition of prothrombinase.

FIG. 8 illustrates the chemical structure of additional various peptides of interest (SEQ ID NO. 7-9) according to the present discovery.

FIGS. 9A and 9B show the inhibitory effect upon the activation of factor VIII (FIG. 9A) and factor V (FIG. 9B) by a particular peptide of interest.

FIG. 10A is a graph illustrating the inhibitory effect of various peptides of interest (SEQ ID NO. 7-9, 11).

FIG. 10B is a graph illustrating the inhibitory effect of a peptide of interest (SEQ ID NO. 9) upon the reaction kinetics of prothrombinase.

FIG. 11A is a graph illustrating the inhibitory effect of a particular peptide of interest (SEQ ID NO. 9) upon intrinsic tenase.

FIG. 11B and its inset panel are graphs illustrating the inhibitory effect of increasing concentration of a particular peptide of interest (SEQ ID NO. 9).

FIG. 12 is a graph illustrating the effect upon clotting time by a number of peptides of interest (SEQ ID NO. 7-9, 11).

FIG. 13 is a graph illustrating the effect upon clotting time by a number of peptides of interest (SEQ ID NO. 7-9, 11).

### DETAILED DESCRIPTION

The process of blood coagulation can be separated into three phases: initiation, propagation and termination. Initiation begins when tissue factor (TF) is released into the bloodstream. TF activates factor X to factor Xa. Factor Xa cleaves prothrombin to form thrombin, which is a major component in the coagulation process. Thrombin activates factor V to factor Va. Factors Xa and Va bind to each other and to prothrombin to form a prothrombinase complex that, in the presence of calcium (Ca²⁺) and phospholipids, accelerates the cleavage of prothrombin to thrombin. The essence of propagation is this rapid creation of thrombin. Termination involves the inactivation of the coagulation process.

The TF pathway is thought to proceed by assembly of three distinct complexes. The first is the extrinsic tenase (factor VIIa and the membrane-bound cofactor TF), which assembles when TF, normally sequestered from contact with the plasma portion of blood, encounters circulating factor VIIa because of an injury to the vasculature. Factor IXa assembles with factor VIIIa to form the intrinsic tenase complex, which produces additional factor Xa. Free factor Xa assembles with factor Va into the prothrombinase complex on the cell surface, which is the activator of prothrombin to α-thrombin. At high concentrations of TF, enough factor Xa and prothrombinase is produced by the extrinsic tenase alone (independent of the intrinsic tenase) to overcome inhibition by tissue factor pathway inhibitor (TFPI) and AT-III facilitating thrombin generation at levels capable of sustaining hemostasis. However, following initiation at lower TF concentrations, factor Xa generated by the extrinsic tenase is insufficient to maintain an ongoing hemostatic response. Under these conditions, the intrinsic tenase complex provides the additional factor Xa required to maintain thrombin generation. The importance of the prothrombinase, extrinsic and intrinsic tenase complexes is underscored by the observation that deficiencies in factor VII, factor VIII, factor IX, and factor V are invariably associated with hemorrhagic tendencies.

The prothrombinase complex, which is composed of the non-enzymatic cofactor, factor Va, the enzyme, factor Xa, and the substrate, prothrombin, associated on a cell membrane-surface in the presence of Ca²⁺ ions, is responsible for α-thrombin formation during blood coagulation. The prothrombinase complex catalyzes the activation of prothrombin approximately 300,000-times more efficiently than factor Xa alone. The increase in the catalytic efficiency of prothrombinase as compared to factor Xa alone arises from a decrease in the Kₘ and an increase in the k_{cat} of the enzyme. The procofactor, factor V, does not interact with the components of prothrombinase. Proteolytic processing of factor V by thrombin at Arg⁷⁰⁹, Arg¹⁰¹⁸, and Arg¹⁵⁴⁵, resulting in the production of the active cofactor, factor Va, that consists of a heavy chain (Mᵣ 105,000) component and a light chain (Mᵣ 74,000) component, is required for the interaction of the cofactor with the members of prothrombinase. In contrast, proteolytic inactivation of factor Va by activated protein C (APC) results in its inactivation because of the inability of the cleaved cofactor to interact with factor Xa and prothrombin.

Earlier data have demonstrated that while both chains of factor Va are required for the interaction with factor Xa, only the heavy chain of the cofactor binds prothrombin. Cleavage of factor Va by APC at Arg⁵⁰⁶/Arg⁶⁷⁹ results in a 10-fold decrease in the affinity of the molecule for factor Xa and the elimination of its interaction with prothrombin. Subsequent cleavage at Arg³⁰⁶, which is lipid-dependent, completely abolishes the ability of the cofactor to interact with factor Xa. Prothrombin and thrombin have two distinct electropositive binding exosites (anion binding exosite I, ABE-I, and anion binding exosite II, ABE-II) that are responsible for the functions of the molecules. ABE-I has been involved in binding to thrombomodulin, fibrinogen, heparin cofactor II, PAR1, and the COOH-terminal hirudin peptides. ABE-II was found to be involved in the interaction with heparin cofactor II, protease nexin, and antithrombin III. While the involvement of ABE-I of prothrombin in the productive interaction with factor Va within prothrombinase has been demonstrated, some data also suggested that ABE-II of the molecule is also involved in the activation of factors V and VIII. Proexosite I of prothrombin, which is present in a low affinity state on the molecule, is fully exposed following activation and formation of thrombin, and the affinity for its ligands increases by approximately 100 times. The procofactor, factor V, was found to interact with immobilized thrombin through ABE-I but with a lower affinity than factor Va. As a consequence, there has been no interaction reported between prothrombin and factor V since the binding sites involved in the interaction between the two molecules are most likely hidden within their core and at least one of the two molecules must be activated and a portion or an entire exosite must be exposed for the binary interaction to occur.

Factor Va is required for the presentation of the substrate (prothrombin) to the enzyme (factor Xa). There is evidence suggesting that incorporation of factor Va into prothrombinase and its interaction with factor Xa and prothrombin, does not significantly alter the catalytic triad of the enzyme. It has been suggested that upon the interaction with factor Va, factor Xa expresses cryptic exosites for prothrombin, which in turn appear to be largely responsible for the increase in the catalytic efficiency of the enzyme within prothrombinase. These latter studies were performed with specific inhibitors of factor Xa that interact with the enzyme at precise sites remote from its active site. However, several laboratories have demonstrated that prothrombin and thrombin bind to the isolated heavy chain of the cofactor in a calcium-independent manner through ABE-I.

While a binding site for factor Xa has been recently identified on the heavy chain of cofactor Va, the specific site(s) on the heavy chain of the cofactor that interact with thrombin and prothrombin remain to be identified. Accordingly, there exists a need for identification of the specific site(s) on the heavy chain component of factor Va.

An informative review of the clotting mechanism is provided by Kalafatis et al., 1997, “Regulation of Clotting Factors,” Critical Reviews in Eukaryotic Gene Expression, 7(3): 241-280; herein incorporated by reference.

In accordance with the present discovery, it has been demonstrated that using various proteolytic enzymes, the carboxyl-terminal portion of the heavy chain of factor Va (residues 680-709 and designated as SEQ ID NO. 1) is responsible for the interaction of factor Va with one or both components of prothrombinase. This amino acid region is highly acidic in nature. This amino acid region is believed to possess residues that are directly involved in the interaction of the cofactor with positively charged amino acids provided by one of the protein components of prothrombinase. Further, it has been demonstrated that a binding site for prothrombin is located on the last thirteen amino acids of the factor Va heavy chain. As described in greater detail herein, the specific amino acid residues from the acidic COOH-terminal region of factor Va heavy chain that are important for cofactor function and the molecular mechanisms underlying their contribution are identified.

Specifically, a functionally important cluster of amino acids is located on the COOH-terminal portion of the heavy chain of factor Va, between amino acid residues 680-709. To ascertain the importance of this region for cofactor activity, five overlapping peptides representing this amino acid stretch (10 amino acids each, HC1-HC5, designated herein as SEQ ID NO. 2-6, respectively) were synthesized and tested for inhibition of prothrombinase assembly and function. Two peptides, HC3 (spanning amino acid region 690-699) (SEQ ID NO. 4) and HC4 (containing amino acid residues 695-704) (SEQ ID NO. 5) were found to be potent inhibitors of prothrombinase activity with IC₅₀'s of about 12 μM and about 10 μM, respectively. The two peptides were unable to interfere with the binding of factor Va to active-site fluorescently labeled Glu-Gly-Arg human factor Xa ([OG488]-EGR-hXa), and kinetic analyses showed that HC3 and HC4 are competitive inhibitors of prothrombinase with respect to prothrombin with K_{I}'s of approximately 6.3 μM and approximately 5.3 μM, respectively. These data suggest that the peptides inhibit prothrombinase because they interfere with the incorporation of prothrombin into prothrombinase. The shared amino acid motif between HC3 and HC4 is composed of Asp⁶⁹⁵-Tyr⁶⁹⁶-Asp⁶⁹⁷-Tyr⁹⁸-Gln⁶⁹⁹ (DYDYQ) (SEQ ID NO. 11). A pentapeptide with this sequence inhibited both prothrombinase function with an IC₅₀ of 1.6 μM (with a K_{D} for prothrombin of 850 nM), and activation of factor V by thrombin. Peptides HC3 (SEQ ID NO. 4), HC4 (SEQ ID NO. 5), and DYDYQ (SEQ ID NO. 11) were also found to interact with immobilized thrombin. Thus, the amino acid sequence 695-699 of factor Va heavy is significant for procofactor activation and is required for optimum prothrombinase function. The amino acid sequence 695-699 of factor Va heavy chain has also been discovered to be crucial for both factor Va cofactor activity and cleavage of factor V by thrombin.

It has also been discovered that amino acids 695-698 (SEQ ID NO. 10) of factor Va heavy chain are crucial for both factor Va cofactor activity and cleavage of factor V by thrombin at Arg⁷⁰⁹ and activation. These four amino acids are part of the acidic COOH-terminal portion of factor Va heavy chain (amino acid residues 680-709) (SEQ ID NO. 1). This entire sequence is not conserved among species with only 7 amino acids being identical (7/30˜23%). By mutating residues 695-698 a recombinant factor V molecule has been obtained which is impaired in its activation by thrombin and is deficient in its clotting activity. This molecule has also impaired cofactor activity in a prothrombinase assay using purified reagents and saturating concentrations of factor Xa. It is remarkable that such a dramatic effect on both factor V activation and cofactor function has been demonstrated, by merely changing four amino acids among the 2196 residues in factor V. However, a similar dramatic reduction of clotting and intrinsic tenase activity as well as a decrease in thrombin cleavage efficiency was observed when several important tyrosine residues adjacent to thrombin activating cleavage sites were mutated to phenylalanine in recombinant human factor VII.

And, in another aspect according to the present discovery, it has been discovered that sulfonation of certain amino acids of the peptides of interest results in even greater inhibitory effects upon thrombinase. Thus, the amino acid motif DYDYQ (Asp-Tyr-Asp-Tyr-Gln) (SEQ ID NO. 11) described herein appears to be a good substrate for sulfation and can also mediate a productive interaction with ABE-I of thrombin. It is important to note that while the amino acid motif DYQ (Asp-Tyr-Gln) is conserved among species, the preceding two amino acids of this sequence vary considerably among them. Since it has been shown that there is a difference in prothrombinase efficiency when mixing bovine prothrombin with human prothrombinase as compared with activation of human prothrombin by human prothrombinase, it is believed that while the amino acid sequence DYDYQ of human factor Va provides a binding site for the prothrombin molecule, the first two amino acids of this motif may be required for species specificity recognition. However, it must be noted that the possibility that region DYDYQ of the cofactor modulates a remote portion of factor Va that in turn is responsible for the interaction of factor Va with thrombin and prothrombin cannot be excluded. Accordingly, the present discovery also includes a peptide having an amino acid sequence DYDY (SEQ ID NO. 10) or DYDYQ (SEQ ID NO. 11) in which at least one of the Y amino acids is sulfonated, e.g. DY(—SO₃)DY (SEQ ID NO. 12), DYDY(—SO₃) (SEQ ID NO. 13), DY(—SO₃)DY(—SO₃) (SEQ ID NO. 14), DY(—SO₃)DYQ (SEQ ID NO. 7), DYDY(—SO₃)Q (SEQ ID NO. 8), DY(—SO₃)DY(—SO₃)Q (SEQ ID NO. 9), or combinations of these sequences.

A 42 amino acid peptide (N42R) (SEQ ID NO. 23) from the middle portion of the factor Va heavy chain (representing residues 307-351 of factor V) produces a cofactor effect on factor Xa, increasing the catalytic efficiency of the enzyme by several-fold. Similarly, a nine amino acid peptide (AP4′, residues 323-331) (SEQ ID NO. 24) and a five amino acid peptide (E5A, residues 323-327) (SEQ ID NO. 25) from N42R also generated a cofactor effect when incubated with factor Xa alone. Nonetheless, the effect of all these peptides on the enzyme was not as pronounced as with intact factor Va. All these peptides contain a portion or the entire binding site of factor Va heavy chain for factor Xa. Thus, binding of the cofactor or of its isolated binding domains to factor Xa most likely results in the exposure of specific binding exosites on the enzyme necessary for prothrombin docking as suggested. However, the magnitude of the effect observed on factor Xa was several fold smaller in the presence of the peptides when compared to the effect produced by the entire factor Va molecule. While the extent of the cofactor effect on factor Xa may be dependent on the size of the molecule and/or the multiple points of contact from factor Va that participate to the binding to the enzyme, it is also possible that expression of the hidden exosite for prothrombin on factor Xa may not be enough by itself to account for the dramatic increase in the catalytic efficiency of factor Xa within prothrombinase when compared with factor Xa alone.

While the critical role of factor Va for timely and specific prothrombin activation by prothrombinase has been long established, the molecular mechanism by which factor Va accelerates the catalytic efficiency of factor Xa upon prothrombinase assembly remains an enigma. Several studies based on experiments using either, inhibitors of prothrombinase that interact with factor Xa at sites remote from its active site, or active-site inhibited thrombin, have offered a litany of arguments in favor of the hypothesis that incorporation of factor Va into prothrombinase only results in the exposure of cryptic exosites on factor Xa that facilitates its interaction with prothrombin. Because complete inhibition of prothrombinase occurred when using these competitive inhibitors without any interference with the active site of the enzyme, it was also concluded that the exposed cryptic exosites on factor Xa alone, following its interaction with factor Va, may account for the substrate specificity of prothrombinase. It is believed that factor Va heavy chain interacts directly with prothrombin through ABE-I and probably ABE-II. These latter conclusions are also supported by the fact that no interaction between bovine prothrombin and factor Xa could be detected in the absence of factor Va. A direct interaction between factor Va and active-site labeled meizothrombin on the membrane surface has also been demonstrated. More recently, the direct involvement of ABE-I of prothrombin-1 (prothrombin molecule lacking the Gla and Kringle-1 domains) with factor Va has been demonstrated. These latter studies showed that Arg⁶² and Lys⁶⁵ of the B chain of thrombin (Arg⁶⁷ and Lys⁷⁰ chymotrypsin numbering) were the major contributing amino acid residues from ABE-I of prothrombin to prothrombinase activity. All these data suggest that a significant conformational transition of the proteinase domain of the prothrombin molecule occurs upon its interaction with factor Va. It was thus hypothesized that factor Va may be at least partially responsible for the rearrangement of the prothrombin structure allowing exposure of hidden or non-optimally exposed proteolytic sites required for efficient substrate catalysis. This extensive molecular rearrangement of prothrombin for efficient catalysis at Arg³²⁰ was also suggested following the determination of the crystal structure of prothrombin 2.

In accordance with the present discovery, it is believed that the extensive binding exosite for prothrombin which provides for prothrombinase specificity and is responsible for the correct docking of the substrate in the active site of the enzyme, is most likely provided by amino acids belonging to both the carboxyl-terminal portion of factor Va heavy chain and factor Xa. Complete inhibition of either one separately will result in the loss of the catalytic efficiency of prothrombinase. However, it is important to note that there is not enough evidence overall to conclude that ABE-I of prothrombin interacts exclusively with the DYDYQ motif (SEQ ID NO. 11) from factor Va heavy chain. It is possible that the extended surface spanning ABE-I is also responsible for the interaction of the substrate with the cryptic exosite from factor Xa exposed upon its interaction with factor Va. Thus, in all cases (i.e. if ABE-I interacts exclusively with factor Va heavy chain, or exclusively with the cryptic exosite from factor Xa, or with both) it would appear that the role of ABE-I in prothrombinase is dependent on the incorporation of factor Va into the complex as suggested.

In summary, the results of testing described herein illustrate the importance of factor Va for the specificity involved in substrate recognition and cleavage by prothrombinase and specifically, demonstrate that: 1) factor Va binds factor Xa (receptor effect); 2) following binding a conformational transition of the enzyme occurs exposing a portion of the binding exosite(s) for prothrombin (effector effect on factor Xa); and 3) the extended and contiguous prothrombin binding exosite within prothrombinase is completed by a portion of the heavy chain of the cofactor (effector effect on prothrombinase). The latter is required to achieve rates of prothrombin generation observed with prothrombinase. These results thus define the cofactor, factor Va, as being the primary determinant of prothrombinase and thereby orchestrating the spatial rearrangement of substrate and enzyme, which in turn are necessary for specific and efficient catalysis.

In accordance with the present discovery, various peptides and specifically, a pentapeptide containing the amino acid sequence DYDYQ (SEQ ID NO. 11), have been found to significantly inhibit the generation of thrombin and thus, serve as anticoagulants. Other peptides of interest according to the present discovery include, but are not limited to, those peptides containing the amino acid sequence DYDYQ (SEQ ID NO. 11). In addition, the amino acid sequence DYDY (SEQ ID NO. 10), the sulfonated sequences of DYDYQ (SEQ ID NO. 7-9) and DYDY (SEQ ID NO. 12-14) in which at least one of the Y amino acids is sulfonated, have been found to be particularly beneficial as inhibitors of thrombin generation. The single letter abbreviations for various amino acids referred to herein are according to those set forth by IUPAC and detailed in “Nomenclature and Symbolism for Amino Acids and Peptides,” Eur. J. Biochem. 138: 9-37 (1984), herein incorporated by reference. And thus, further peptides of interest include those containing the amino acid sequence DYDY, or the sulfonated sequence DYDY or DYDYQ in which at least one of the Y amino acids is sulfonated. As further explained herein, all of these peptides of interest exhibit significant inhibitory effects upon prothrombinase. The sulfonated peptides have been discovered to exhibit particular and unexpected inhibitory functions. Moreover, the sulfonated peptides exhibit an inhibitory function upon intrinsic tenase.

A characteristic of these various peptides is their IC₅₀ value, which is generally the amount of the peptide which inhibits 50% of the Va cofactor's normal activity. As will be appreciated, a lower value indicates a greater inhibiting effect upon thrombin generation.

Generally, the peptides of interest in accordance with the present discovery exhibit IC₅₀ values of less than about 100 μM, less than about 50 μM, including less than about 40 μM, less than about 30 μM, less than about 20 μM, less than about 15 μM, including about 12 μM and about 10 μM, and including less than about 5 μM, and less than about 2.5 μM and including about 1.6 μM and about 500 nM.

The peptides according to the present discovery may contain amino acids that are non-naturally occurring. Naturally occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. Some examples of non-naturally occurring amino acids are norleucine, norvaline, alloisoleucine, homoarginine, thiaproline, dehydroproline, hydroxyproline, homoserine, cyclohexylglycine, -amino-n-butyric acid, cyclohexylalanine, aminophenylbutyric acid, phenylalanines substituted at the ortho, meta, or paraposition of the phenyl moiety with one or two of the following, a (C₁-C₄) alkyl, (C₁-C₄) alkoxy, halogen, or nitro groups or substituted with a methylenedioxy group, 2- and 3-thienylalanine, -2- and 3-furanylalanine, -2-, 3-, and 4-pyridylalanine, -(benzothienyl-2- and 3-yl)alanine, -(1- and 2-naphthyl)alanine, O-alkylated derivates of serine, threonine, or tyrosine, S-alkylated cysteine, the O-sulfate ester of tyrosine, 3,5-diiodotyrosine and the D-isomers of the naturally occurring amino acids. These and any other non-naturally occurring amino acids can be included in the inventive peptides so long as they do not adversely affect the anticoagulation activity of these peptides, or provide adverse side effects, in any significant way.

Another modification that may be embodied in the peptides according to the present discovery is that they may contain one or more D-amino acids, rather than the L-amino acids that are found in naturally-occurring proteins. L and D refer to the stereochemistry of the amino acids. More specifically, L and D refer to the absolute configuration of the four atoms attached to the a carbon atom of the amino acid. L and D are designations well known to those skilled in the art. Peptide bonds involving D amino acids are less susceptible to cleavage by proteases than are peptide bonds involving L amino acids. Peptides containing D amino acids, therefore, may have a longer half life in vivo than peptides that do not contain D amino acids.

Also included in the present discovery are peptides containing one or more non-hydrolyzable bonds between adjacent amino acids. Such non-hydrolyzable bonds are different than the amide linkages between the α-amino group of one amino acid and the α-carboxyl group of a second amino acid (—CO—NH—). Such non-hydrolyzable bonds may include, for example, —CH₂NH—, —CH₂S—, —CH₂O—, —CH₂CH₂—, —CH═CH—(cis and trans), —COCH₂—, —CH(OH)CH₂—, —CH₂SO—, —CH₂SO₂—, and —CH(CN)NH—. These bonds can be formed by methods known in the art. The following references describe preparation of peptide analogs which include these alternative-linking moieties: Spatola, March 1983, “Peptide Backbone Modifications” (general review) Vega Data, Vol. 1, Issue 3; Spatola, 1983, in Chemistry and Biochemistry of Amino Acids, Peptides and Proteins (general review), B. Weinstein editor, Marcel Dekker, New York, p. 267; Morley, 1980, Trends Pharm. Sci., 468:463-468 (general review); Hudson, et al., 1979, Int. J. Pept. Prot. Res. 14:177-185 (—CH₂NH—, —CH₂CH₂—); Spatola, et al., 1986, Life Sci. 38:1243-1249 (—CH₂S—); Hann, 1982, Chem. Soc. Perkin Trans. I, pp. 307-314 (—CH—CH—, cis and trans); Almquist, et al., 1980, J. Med. Chem. 23:1392-1398 (—COCH₂—); Jennings-White, et al., 1982, Tetrahedron Lett. 23:2533 (—COCH₂—); Szelke, et al., 1982, European Application EP 45665; CA:97:39405 (—CH(OH)CH₂—); Holladay, et al., 1983, Tetrahedron Lett 24:4401-4404 (—CH(OH)CH₂—); and Hruby, 1982, Life Sci. 31:189-199 (—CH₂S—); all of which are hereby incorporated by reference.

Another modification that may be contained in the peptides according to the present discovery are modifications that result in peptides called “constrained peptides” (including cyclized peptides). One example of a cyclized peptide is a peptide that has at least one cysteine amino acid at or near each end of the peptide. Through formation of intramolecular disulfide bridges between the cysteines, the peptide becomes cyclized. Such constrained peptides may be generated by methods known in the art (Rizo and Gierasch, 1992, Annu Rev Biochem, 61:387-418), herein incorporated by reference, and are more resistant to proteases in vivo than are peptides of the same amino acid sequence that are not cyclized.

The present discovery also includes the use of peptide analogue(s) in place of, or in addition to, the peptides described herein. A peptide analogue as referred to herein refers to a compound that is capable of mimicking or antagonizing the biological action(s) of a parent or natural peptide. An example of a peptide analogue is a peptidomimetic. Generally, a peptide analogue as used herein is a compound that mimics the critical features of the molecular recognition process of the parent peptide and thereby blocks or reproduces the action of the peptide. An example of a non-peptide peptidomimetic agonist for a peptide receptor system is morphine, which mimics the opioid peptides. A peptide analogue can also include any of the previously noted non-naturally occurring peptides, stereoisomers, peptides containing one or more non-hydrolysable bonds between adjacent amino acids, constrained peptides, or equivalents thereof. Similarly, each of the sequence identifiers noted herein include and encompass conservatively modified variants thereof.

Methods of Synthesizing Peptides

A wide variety of different techniques are known for making peptide segments, and any such method can be used in making the peptides according to the present discovery.

Most often, synthesis of peptides involves chemical synthesis and can include subsequent treatment under oxidizing conditions appropriate to obtain the native conformation, that is, the correct disulfide bond linkages. This can be accomplished using methodologies well known to those skilled in the art (Kelly and Winkler, 1990, in Genetic Engineering Principles and Methods, vol. 12, J. K. Setlow editor, Plenum Press, New York, pp. 1-19; Stewart and Young, 1984, Solid Phase Peptide Synthesis, Pierce Chemical Co., Rockford, Ill.), herein incorporated by reference. One such method is described below.

In one embodiment, peptides in accordance with the present discovery can be prepared using solid phase synthesis (Merrifield, 1964, J Amer Chem Soc, 85:2149; Houghten, 1985, Proc Natl Acad Sci USA, 82:5131-5), herein incorporated by reference. Solid phase synthesis can begin at the C-terminus of the putative peptide by coupling a protected amino acid to a suitable resin. In this synthesis, the carboxyl terminal amino acid, with its α-amino group suitably protected, can be coupled to a chloromethylated polystyrene resin. After removal of the α-amino protecting group with, for example, trifluoroacetic acid (TFA) in methylene chloride and neutralizing in, for example TEA, the next cycle in the synthesis can proceed.

The remaining α-amino- and, if necessary, side-chain-protected amino acids can then be coupled sequentially in the desired order by condensation to obtain an intermediate compound connected to the resin. Alternatively, some amino acids may be coupled to one another forming a peptide prior to addition of the peptide to the growing solid phase peptide chain. The condensation between two amino acids, or an amino acid and a peptide, or a peptide and a peptide can be carried out according to the usual condensation methods such as azide method, mixed acid anhydride method, DCC (dicyclohexylcarbodiimide) method, active ester method (p-nitrophenyl ester method, BOP [benzotriazole-1-yl-oxy-tris (dimethylamino) phosphonium hexafluorophosphate] method, N-hydroxysuccinic acid imido ester method, etc.), and Woodward reagent K method. In the case of elongating the peptide chain in the solid phase method, the peptide can be attached to an insoluble carrier at the C-terminal amino acid. For insoluble carriers, those which react with the carboxy group of the C-terminal amino acid to form a bond which is readily cleaved later, for example, halomethyl resin such as chloromethyl resin and bromomethyl resin, hydroxymethyl resin, aminomethyl resin, benzhydrylamine resin, and t-alkyloxycarbonyl-hydrazide resin can be used.

Common to chemical synthesis of peptides is the protection of the reactive side-chain R groups of the various amino acid moieties with suitable protecting groups at that site until the group is ultimately removed after the chain has been completely assembled. Also common is the protection of the α-amino group on an amino acid or a fragment while that entity reacts at the carboxyl group followed by the selective removal of the α-amino-protecting group to allow subsequent reaction to take place at that location. Accordingly, it is common that, as a step in the synthesis, an intermediate compound is produced which includes each of the amino acid residues located in the desired sequence in the peptide chain with various of these residues having side-chain protecting groups. These protecting groups are then commonly removed substantially at the same time so as to produce the desired resultant product following purification.

The applicable protective groups for protecting the reactive amino side-chain groups of the various amino acid moieties are exemplified by benzyloxycarbonyl (abbreviated Z), isonicotinyloxycarbonyl (iNOC), O-chlorobenzyloxycarbonyl [Z(NO₂], p-methoxybenzyloxycarbonyl [Z(OMe)], t-butoxycarbonyl, (Boc), t-amyioxycarbonyl (Aoc), isobornyloxycarbonyl, adamatyloxycarbonyl, 2-(4-biphenyl)-2-propyloxycarbonyl (Bpoc), 9-fluorenylmethoxycarbonyl (Fmoc), methylsulfonyiethoxycarbonyl (Msc), trifluoroacetyl, phthalyl, formyl, 2-nitrophenylsulphenyl (NPS), diphenylphosphinothioyl (Ppt), dimethylophosphinothioyl (Mpt) and the like.

As protective groups for carboxy groups there can be exemplified, for example, benzyl ester (OBzI), cyclohexyl ester (Chx), 4-nitrobenzyl ester (ONb), t-butyl ester (Obut), 4-pyridylmethyl ester (OPic), and the like. It is desirable that specific amino acids such as arginine, cysteine, and serine possessing a functional group other than amino and carboxyl groups are protected by a suitable protective group as occasion demands. For example, the guanidino group in arginine may be protected with nitro, p-toluenesulfonyl, benzyloxycarbonyl, adamantyloxycarbonyl, p-methoxybenzenesulfonyl, 4-methoxy-2,6-dimethylbenzenesulfonyl (Mds), 1,3,5-trimethylphenysulfonyl (Mts), and the like. The thiol group in cysteine may be protected with p-methoxybenzyl, triphenylmethyl, acetylaminomethyl ethylcarbamoyl, 4-methylbenzyl, 2,4,6-trimethyl-benzyl (Tmb) etc, and the hydroxyl group in the serine can be protected with benzyl, t-butyl, acetyl, tetrahydropyranyl etc.

Stewart and Young, “Solid Phase Peptide Synthesis,” Pierce Chemical Co., Rockford, Ill., 1984, herein incorporated by reference, provides detailed information regarding procedures for preparing peptides. Protection of α-amino groups is described on pages 14-18, and side-chain blockage is described on pages 18-28. A table of protecting groups for amine, hydroxyl and sulfhydryl functions is provided on pages 149-151.

After the desired amino acid sequence has been completed, the intermediate peptide can be removed from the resin support by treatment with a reagent, such as liquid HF and one or more thio-containing scavengers, which not only cleaves the peptide from the resin, but also cleaves all the remaining side-chain protecting groups. Following HF cleavage, the protein sequence can be washed with ether, transferred to a large volume of dilute acetic acid, and stirred at pH adjusted to about 8.0 with ammonium hydroxide.

Preferably, in order to avoid alkylation of residues in the peptide, (for example, alkylation of methionine, cysteine, and tyrosine residues) a thio-cresol and cresol scavenger mixture can be used. The resin can be washed with ether, and immediately transferred to a large volume of dilute acetic acid to solubilize and minimize intermolecular cross-linking. A 250 μM peptide concentration can be diluted in about 2 liters of 0.1 M acetic acid solution. The solution can then be stirred and its pH adjusted to about 8.0 using ammonium hydroxide. Upon pH adjustment, the peptide takes its desired conformational arrangement.

Kunitz domains (i.e., functional sites) can be made either by chemical synthesis, described above, or by semisynthesis. The chemical synthesis or semisynthesis methods of making allow the possibility of modified amino acid residues to be incorporated. This has been carried out for Kunitz domains and related proteins as previously described in Beckmann, et al., 1988, Eur J Biochem, 176:675-82; and Bigler, et al., 1993, Protein Sci, 2:786-99, herein incorporated by reference.

Thrombin Inhibitors—Therapeutic Uses—Methods of Using

Anticoagulant therapy is indicated for the treatment and prevention of a variety of thrombotic conditions, particularly coronary artery and cerebrovascular disease. Those experienced in this field are readily aware of the circumstances requiring anticoagulant therapy. The term “patient” used herein refers to mammals such as primates, including humans, sheep, horses, cattle, pigs, dogs, cats, rats, and mice.

The peptides according to the present discovery can be used as medicines to prevent thrombotic disorders resulting from the formation of blood clots that obstruct blood vessels. There are a wide variety of conditions that predispose or lead to thrombosis. Some of these conditions are coronary artery disease, valvular heart disease, stable and unstable angina, myocardial infarction, atrial fibrillation and stroke. Other subjects at risk for thrombosis are those undergoing coronary angioplasty, those with coronary artery bypass grafts or prosthetic heart valves, those with high cholesterol levels in the blood, those that have catheters inserted into blood vessels, women taking oral contraceptives or individuals with genetic disorders causing a predisposition to blood coagulation. Additional conditions for which the present peptides can be used, include, but are not limited to, deep vein thrombosis, pulmonary embolism, thrombophlebitis, arterial occlusion from thrombosis or embolism, arterial reocclusion during or after angioplasty or thrombolysis, restenosis following arterial injury or invasive cardiological procedures, postoperative venous thrombosis or embolism, acute or chronic atherosclerosis, stroke, myocardial infarction, cancer and metastasis, and neurodegenerative diseases. The peptides or pharmaceutical compositions containing such may also be used as anticoagulants in extracorporeal blood circuits, as necessary in dialysis and surgery. The peptides or pharmaceutical compositions may also be used as in vitro anticoagulants.

Thrombin inhibition is useful not only in the anticoagulant therapy of individuals having thrombotic conditions, but is useful whenever inhibition of blood coagulation is required such as to prevent coagulation of stored whole blood and to prevent coagulation in other biological samples for testing or storage. Thus, the thrombin inhibitors according to the present discovery can be added to or contacted with any medium containing or suspected of containing thrombin and in which it is desired that blood coagulation be inhibited, e.g., when contacting the mammal's blood with material selected from the group consisting of vascular grafts, stents, orthopedic prosthesis, cardiac prosthesis, and extracorporeal circulation systems.

The peptides or pharmaceutical compositions containing such, generally referred to herein as “thrombin inhibitors,” are useful for treating or preventing venous thromboembolism (e.g. obstruction or occlusion of a vein by a detached thrombus; obstruction or occlusion of a lung artery by a detached thrombus), cardiogenic thromboembolism (e.g. obstruction or occlusion of the heart by a detached thrombus), arterial thrombosis (e.g. formation of a thrombus within an artery that may cause infarction of tissue supplied by the artery), atherosclerosis (e.g. arteriosclerosis characterized by irregularly distributed lipid deposits) in mammals, and for lowering the propensity of devices that come into contact with blood to clot blood.

Examples of venous thromboembolism which may be treated or prevented with the peptides of the present discovery include obstruction of a vein, obstruction of a lung artery (pulmonary embolism), deep vein thrombosis, thrombosis associated with cancer and cancer chemotherapy, thrombosis inherited with thrombophilic diseases such as Protein C deficiency, Protein S deficiency, antithrombin III deficiency, and Factor V Leiden, and thrombosis resulting from acquired thrombophilic disorders such as systemic lupus erythematosus (inflammatory connective tissue disease). Also with regard to venous thromboembolism, the peptides and compositions of the present discovery are useful for maintaining patency of indwelling catheters.

Examples of cardiogenic thromboembolism which may be treated or prevented with the peptides of the present discovery include thromboembolic stroke (detached thrombus causing neurological affliction related to impaired cerebral blood supply), cardiogenic thromboembolism associated with atrial fibrillation (rapid, irregular twitching of upper heart chamber muscular fibrils), cardiogenic thromboembolism associated with prosthetic heart valves such as mechanical heart valves, and cardiogenic thromboembolism associated with heart disease.

Examples of arterial thrombosis include unstable angina (severe constrictive pain in chest of coronary origin), myocardial infarction (heart muscle cell death resulting from insufficient blood supply), ischemic heart disease (local anemia due to obstruction (such as by arterial narrowing) of blood supply), reocclusion during or after percutaneous transluminal coronary angioplasty, restenosis after percutaneous transluminal coronary angioplasty, occlusion of coronary artery bypass grafts, and occlusive cerebrovascular disease. Also with regard to arterial thrombosis, the peptides of the present discovery are useful for maintaining patency in arteriovenous cannulas.

The peptides of the present discovery can be administered to humans in an amount that prevents formation of unwanted blood clots. Generally, such an amount will be from about 0.01 to 1000 mg/kg per day, more preferably from about 0.1 to 100 mg/kg per day, most preferably from about 1 to 10 mg/kg per day. The amount of peptide that prevents unwanted blood clots, however, will vary with the IC₅₀ of the peptide as well as with the half-life of the peptide in the body. The amount of peptide that prevents unwanted blood clots will also vary with the particular condition being treated, the age and physical condition of the subject being treated, the severity of the condition, the duration of the treatment, the nature of the concurrent therapy (if any), the specific route of administration and like factors within the knowledge and expertise of the health practitioner.

In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of peptides.

Thus, it will be understood that the peptide coagulation inhibitors of the present discovery can be used to inhibit blood clotting and thrombotic diseases in subjects at risk of developing such disorders.

When administered to a subject, the peptides according to the present discovery can be given as pharmaceutically-acceptable compositions. Such compositions may routinely contain salt, buffering agents, preservatives, adjuvants, other vehicles and, optionally, other therapeutic agents. The peptides may be optionally combined with a pharmaceutically-acceptable carrier. As will be appreciated by those skilled in the art, the peptides can be delivered or incorporated in a pharmaceutical composition in a protected, i.e. chemically or physically, form.

The peptides, generally speaking, may be administered using any mode that is medically acceptable, meaning any mode that produces effective levels of the active peptides without causing clinically unacceptable adverse effects. Such modes of administration include parenteral routes (e.g., intravenous, intra-arterial, subcutaneous, intramuscular, mucosal or infusion), but may also include oral, rectal, topical, nasal or intradermal routes. Other delivery systems can include time-release, delayed release or sustained release delivery systems. Such systems can avoid repeated administrations, increasing convenience to the subject and the physician. Many types of release delivery systems are available and known to those of ordinary skill in the art.

Compositions suitable for parenteral administration are preferred and conveniently comprise a sterile aqueous or oleaginous preparation of the peptide, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa., herein incorporated by reference. The pharmaceutical compositions may conveniently be presented in unit dosage form and may be prepared by any of the methods well-known in the art of pharmacy.

Specifically, the thrombin inhibitors of the present discovery can be administered in such oral forms as tablets, capsules (each of which includes sustained release or timed release formulations), pills, powders, granules, elixers, tinctures, suspensions, syrups, and emulsions. Likewise, they may be administered in intravenous (bolus or infusion), intraperitoneal, subcutaneous, or intramuscular form, all using forms well known to those of ordinary skill in the pharmaceutical arts. An effective but non-toxic amount of the compound desired can be employed as an anti-aggregation agent. For treating ocular build up of fibrin, the compounds may be administered intraocularly or topically as well as orally or parenterally.

The thrombin inhibitors can be administered in the form of a depot injection or implant preparation which may be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers or synthetic silicones, for example, SILASTIC, silicone rubber or other polymers manufactured by the Dow-Corning Corporation.

The thrombin inhibitors can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The thrombin inhibitors may also be delivered by the use of monoclonal antibodies as individual carriers to which the compound molecules are coupled. The thrombin inhibitors may also be coupled with soluble polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the thrombin inhibitors may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The compounds can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

The thrombin inhibitors are typically administered as active ingredients in admixture with suitable pharmaceutical diluents, excipients or carriers (collectively referred to herein as “carrier” materials) suitably selected with respect to the intended form of administration, that is, oral tablets, capsules, elixers, syrups and the like, and consistent with convention pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch methyl cellulose, agar, bentonite, xanthan gum and the like.

The present discovery may be better understood by reference to the following examples, which serve to illustrate but not to limit the present discovery.

Abbreviations:

APC, activated-protein C;

HEPES, N-[2-Hydroxyethyl]piperazine-N′-2-ethanesulfonic acid;

Tris, Tris[hydroxymethyl]aminomethane;

DFP, diisopropyl-fluorophosphate;

PEG, polyethylene glycol Mᵣ 8000; OPD, O-phenylenediamine dihydrochloride;

Phe-Pro-Arg-ck, D-Phenylalanyl-L-prolyl-L-Arginine chloromethyl ketone (FPRck);

ATA-FPRck, N^{α}-[(acetylthio)acetyl]-Phe-Pro-Argck;

PS, L-α-phosphatidylserine; PC, L-α-phosphatidylcholine;

PCPS, small unilamellar phospholipids vesicles composed of 75% PC and 25% PS (w/w);

DAPA, dansylarginine-N-(3-ethyl-1,5-pentanediyl)amide;

[OG₄₈₈]-EGR-hXa, human factor Xa blocked in the active site with glutamylglycinylarginyl chloromethyl ketone labeled with Oregon Green 488;

HPLC, high-performance liquid chromatography;

LC/MS; Liquid Chromatography/Mass Spectrometry;

DMEM, Dulbecco's modified Eagle's medium; FBS, fetal bovine serum;

SDS-PAGE, Sodium dodecyl sulfate polyacrylamide gel electrophoresis;

ELISA, enzyme-linked immunosorbent assay; PVDF, polyvinylidene difluoride;

ABE-I, anion binding exosite I; ABE-II, anion binding exosite II; factor V^{2K2F}, quadruple mutant, recombinant human factor V with the mutations D⁶⁹⁵→K, Y⁶⁹⁶→F, D⁶⁹⁷→K, and Y⁶⁹⁸→F;

factor Va_{IIa}^{2K2F}, quadruple mutant activated with thrombin;

factor Va_{RVV}^{2K2F}, quadruple mutant activated with RV-V activator;

factor Va_{Xa}^{2K2F}, quadruple mutant activated with factor Xa;

Materials reagents, and proteins: Diisopropyl-fluorophosphate (DFP), O-phenylenediamine (OPD)-dihydrochloride, N-[2-Hydroxyethyl]piperazine-N′-2-ethanesulfonic acid (Hepes), Trizma (Tris base), Coomassie Blue R-250, and factor V-deficient plasma were purchased from Sigma (St. Louis, Mo.). The secondary anti-mouse and anti-sheep IgG coupled to peroxidase were from Southern Biotechnology Associates Inc. (Birmingham, Ala.). L-α-phosphatidylserine (PS) and L-α-phosphatidylcholine (PC) where from Avanti Polar Lipids (Alabaster, Ala.). The chemiluminescent reagent ECL⁺ and Heparin-Sepharose were from AmershamPharmacia Biotech Inc (Piscataway, N.J.). Normal reference plasma and the chromogenic substrate Spectrozyme-TH were from American Diagnostica Inc. (Greenwich, Conn.). The thromboplastin reagent for the clotting assays was purchased from Organon Teknika Corp. (Durham, N.C.). Polyethylene glycol Mr 8000 (PEG) was purchased from J. T. Baker (Danvers, Mass.). The fluorescent thrombin inhibitor dansylarginine-N-(3-ethyl-1,5-pentanediyl)amide (DAPA), N^{a}-[(acetylthio)acetyl]-Phe-Pro-Arg-thrombin (ATA-FPR-thrombin) coupled to agarose through the active site as described, RVV-factor V activator, human APC, human factor Xa, human thrombin, human prothrombin, the monoclonal antibody αhFV#1 coupled to Sepharose, and human factor Xa labeled in the active site with Oregon Green 488 ([OG₄₈₈]-EGR-hXa) as previously described, were from Haematologic Technologies Inc. (Essex Junction, Vt.). The cDNA for factor V was purchased from American Type Tissue Collection (ATCC# 40515 pMT2-V, Manassas Va.). The sequence of this cDNA molecule is identical to the cDNA published by Jenny et al. All restriction enzymes were from New England Biolabs (Beverly, Mass.) and all other molecular biology and tissue culture reagents and media were from Gibco, Invitrogen Corporation (Grand Island, N.Y.) or as indicated. The two monoclonal antibodies to human factor V (against the heavy and light chains of the cofactor, i.e. αHFV_{HC}#17 and αHFV_{LC}#9) were provided by Dr. Kenneth G. Mann (Department of Biochemistry, University of Vermont, Burlington Vt.) and have been extensively characterized. Overlapping peptides from the region 680-709 as well as pentapeptide DYDYQ (SEQ ID NO. 11) were synthesized in the Biotechnology Core of the Cleveland Clinic Foundation (Cleveland, Ohio), purified by high-performance liquid chromatography (HPLC), and characterized by mass-spectrometry as described. Human factor V and factor Va were purified and concentrated using methodologies previously described employing the monoclonal antibody αhFV#1 coupled to Sepharose and Heparin-Sepharose. The cofactor activity of the factor Va preparations was measured by a clotting assay using factor V deficient plasma and standardized to the percentage of control as described. Phospholipids vesicles composed of 75% PC and 25% PS (referred to as PCPS vesicles throughout the manuscript) were prepared as previously described. The concentration of phospholipids vesicles was determined by phosphorous assay as described earlier and is given as the concentration of inorganic phosphate.

Determination of factor V/Va clotting activity of the recombinant molecules. Cofactor activity of wild type and mutant molecules was measured in a clotting assay using factor V deficient plasma prior and after activation by thrombin (15 min, 37° C.) and RVV-V activator (2 hr, 37° C.) as described. The values were standardized to the percentage of control. A linear semi-log graph was constructed using known concentrations of purified factor V (U/ml as a function of clotting time). The assay endpoint was determined by visualization of the fibrin clot. The activity of the factor V/Va solution (U/ml) was determined by extrapolation from the graph. The concentration of the recombinant molecules was determined by a recently described ELISA. Finally, the numbers were combined to obtain the specific activity of the recombinant factor V solutions (U/mg).

Assay measuring thrombin formation. The formation of thrombin was analyzed using the fluorescent thrombin inhibitor DAPA as described using a Perkin Elmer LS-50B Luminescence Spectrometer (Perkin-Elmer LLC, Norwalk Conn.) with λₑₓ=280 nm, λₑₘ=550 nm and a 500 nm long pass filter in the emission beam (Schott KV-500). The buffer used in all cases was composed of 20 mM Hepes, 0.15 M NaCl, 5 mM CaCl₂, pH 7.4 [HBS(Ca²⁺), “assay buffer”]. In all cases peptides were preincubated with factor Va prior to the assay as described in the legend to the figures. The final concentration of factor Va in the mixture was 4 nM with factor Xa at 10 nM, prothrombin at 350 nM, DAPA at 700 nM, in the presence of PCPS vesicles (10 μM). The initial rate of thrombin formation (nM IIa·min⁻¹) was calculated as described during the initial 5-10 sec of the reaction. To verify if the peptides have any effect on the active-site of thrombin, control experiments were performed as follows: a given peptide (at 100 μM) was incubated in the assay buffer containing DAPA (700 nM); the base line was monitored for 30 sec; thrombin (350 nM) was then added to the mixture and the fluorescent intensity resulting from the complexation of DAPA with the active site of thrombin was monitored for 60 sec. The slope of the reaction measuring thrombin formation in the presence of a given peptide during the first 5 sec was calculated and compared to the slope of a reaction obtained in the absence of peptide. It is noteworthy that under the conditions employed the thrombin-DAPA interaction occurred fast and the calculated slope of the reaction was sensitive to all parameters used. However, multiple titrations of the same reaction using various preparations of thrombin and peptide, demonstrated that the peptides do not have any significant effect on the capabilities of thrombin to interact with DAPA. All experiments were performed in triplicates or as indicated. The concentration of each peptide given in the figures and figure legends is their final concentration in the assay. The data were stored using the software FL WinLab (Perkin-Elmer Corp, Norwalk Conn.) and further analyzed and plotted with the software Prizm (GraphPad, San Diego, Calif.). In some cases the data were also analyzed and plotted using DeltaGraph (DeltaPoint, Monterey, Calif.).

Fluorescence Anisotropy measurements. Fluorescence anisotropy of [OG₄₈₈]-EGR-hXa was measured using a Perkin Elmer LS-50B Luminescence Spectrometer in L-format as recently described. Anisotropy measurements were performed in a quartz cuvette under constant stirring (low) with λₑₓ=490 nm, λₑₘ=520 nm with a long pass filter (Schott KV-520) in the emission beam. In all cases, the total addition of peptide did not exceed 10% of the volume of the reaction. The concentration of peptide given in each graph is the final concentration of the peptide in the assay mixture. The data were stored using the software FL WinLab (Perkin-Elmer Corp, Norwalk Conn.) and further analyzed and plotted with the software Prizm (GraphPad, San Diego, Calif.). In some cases the data were also plotted using DeltaGraph (DeltaPoint, Monterey, Calif.).

Mutagenesis and transient expression of recombinant factor V molecules. A quadruple mutation of factor V, pMT2-FV-D⁶⁹⁵Y⁶⁹⁶D⁶⁹⁷Y⁶⁹⁸/K⁶⁹⁵F⁶⁹⁶K⁶⁹⁷F⁶⁹⁸ was synthesized by PCR based method as described recently. First, a double mutant FV-D⁶⁹⁵Y⁶⁹⁶/K⁶⁹⁵F⁶⁹⁶ was made in a small DNA fragment of the factor V cDNA. The mutagenic primers for this double mutant fragment were 5′-GAGTGATGCTAAGTTTGATTACC-3′ (sense) (SEQ ID NO. 15) and 5′-GGTAATCAAACTTAGCATCACTC-3′ (anti-sense) (SEQ ID NO. 16) (underlined letters indicate the mismatch) while the outer primers were 5′-CATGGAGTGACCTTCTCG-3′ (sense) (SEQ ID NO. 17) and 5′-TCATCCAGGAGAACC-3′ (anti-sense) (SEQ ID NO. 18). The amplicon was subcloned in the cloning vector pGEM-T and the nucleotide sequences were verified by DNA sequencing. The plasmid having the double mutation was used as template for synthesizing the quadruple mutant. The mutagenic primers in this case were 5′-GCTAAGTTTAAGTTCCAGAACAGACTGG-3′ (sense) (SEQ ID NO. 19) and 5′-CCAGTCTGTTCTGGAACTTAAACTTAGC-3′ (anti-sense) (SEQ ID NO. 20). The outer primers were the same sense and anti-sense outer primers used in the synthesis of first double mutant. The factor V DNA fragment having all four mutations was subcloned into pGEM-T vector and sequenced. Finally, the DNA fragment was removed from the plasmid by digestion with the restriction enzymes Bsu361 and DraIII Following purification of the insert from the agarose gel, the factor V insert that possess the mutations was re-ligated into the plasmid pMT2-FV, in which the DNA fragment between the Bsu361 and DraIII restriction sites was removed. The ligated plasmids were transformed into DH5α bacterial competent cells. Positive ampicillin resistant clones for pMT2-FV mutants were selected. The correct sequences and orientations of the inserts were established by DNA sequence analysis with factor V-specific primers. The wild type pMT2-FV and mutant pMT2-FV plasmids were isolated from the bacterial culture by the QIA filter plasmid Midi kit (QIAGEN Inc. Valencia, Calif.).

Expression of recombinant wild type and mutant factor V in mammalian cells. COS-7 cells (ATTC, Manassas Va.) or COS-7L, (Invitrogen, Grande Island, N.Y.) were maintained in Dulbecco's modified Eagle's medium (DMEM) supplemented with 10% fetal bovine serum (FBS), 2 mM L-glutamine and antibiotics (100 μg/ml streptomycin and 100 IU/ml penicillin) in a humidified atmosphere of 5% CO₂ and 95% air at 37° C.

The purified plasmids pMT2-FV wild type, and pMT2-FV (K⁶⁹⁵F⁶⁹⁶K⁶⁹⁷F⁶⁹⁸) were used to transfect into COS-7L cells as recently described. Following transfection cells were washed twice with serum free medium and 6-10 ml of conditioned media VP-SFM supplemented with 4 mM of L-glutamine were added. After 24 h and 48 h the harvested media containing recombinant factor V was centrifuged at 4,500 rpm at 4° C. to removed insoluble particles. All control media and solutions containing the recombinant factor V molecules were concentrated using centrifugal ultrafiltration (Centricon YM 30,000). The activity and integrity of the molecules was verified before and after thrombin (and/or RVV-V activator) activation by clotting assays using factor V deficient plasma and by SDS-PAGE followed by western blotting using both monoclonal and polyclonal antibodies. The concentration of the recombinant molecules was determined by an ELISA recently described by our laboratory. Because of slight differences in time of incubation with the substrate, in every experiment a plasma factor V standard (serial dilutions of purified plasma factor V) was run and all values obtained with the recombinant molecules were compared to the plasma factor V standard values within the same 96-well plate. No comparison in concentration was made between recombinant molecules from one plate to another. The determination of the concentration of the recombinant molecules was performed by averaging the value found for each sample run in triplicate.

Measurement of rates of thrombin formation in a prothrombinase assay using purified reagents. All factor V species were activated with thrombin for 15 min at 37° C., or with RVV-V activator for two hours at 37° C. as described followed by the addition of DFP. The factor Va solution was then incubated for an additional 30 min on ice. Control experiments demonstrated that under these conditions no interference of the DFP with the assay could be observed since DFP is readily hydrolyzed in aqueous solution. Factor V was also activated by factor Xa in the presence of phospholipids. Assay mixtures contained PCPS vesicles (20 μM), DAPA (3 μM), various concentrations of recombinant factor Va species, prothrombin (1.4 μM), in 20 mM HEPES, 0.15 M NaCl, 5 mM CaCl₂, pH 7.4. The assay was conducted as recently described by measuring thrombin formation by the change in the absorbance of a chromogenic substrate at 405 monitored with a Molecular Devices THERMOMAX microplate reader (Sunnyvale, Calif.). The initial rates of thrombin generation under the conditions employed were linear and in all experiments no more than 10% of prothrombin was consumed during the initial course of the assay. All data were analyzed with the software Prizm (GraphPad, San Diego, Calif.).

Direct binding of the peptides to thrombin. Thrombin was immobilized onto agarose through the active site as described. Peptide solutions of HC1-HC5 (SEQ ID NO. 2-6, respectively), D13R (SEQ ID NO. 21), DYDYQ (SEQ ID NO. 11), and P15H (SEQ ID NO. 22) were dissolved in water to a given concentration and then diluted in 20 mM Hepes, 0.1 M NaCl, pH 7.4 in a manner that 400 μg was contained in each starting solution. In control experiments it was determined that the maximum amount of peptide that could be retained by the thrombin-agarose column was 400 μg. Since some of the peptides contain aromatic amino acid residues, their concentration was also measured by optical density. Nevertheless, the presence of all peptides including those that do not contain aromatic amino acids (HC1, HC2, and HC5) in the void volume or in the fractions representing the elution of the thrombin-agarose column was verified by LC/MS as detailed below in the Analytical Facility of the Cleveland State University.

Mass spectrometry instruments and conditions. The identity of all peptides found in the flow-through or the elution of the thrombin-agarose column was verified by mass spectrometry. Since the peptides were in a buffer solution, we used Liquid Chromatography/Mass Spectrometry (LC/MS) for their identification. In this procedure peptides are first separated from the salt content of the buffer using an HPLC system (HP 1100, HPLC gradient system, Agilent Technologies, Palo Alto Calif.) with a C18 column (1 mm×15 cm, GraceVydac, Hesperia Calif.) with buffers A (0.3% acetic acid in water) and B (0.3% acetic acid in acetonitrile). The elution of the column was monitored with a Micromass Quatro II ESI-Triple Quadrupole Mass Spectrometer (Waters, Milford Mass.). The data were collected using a Compaq Professional Workstation AP200 (Hewlett-Packard, Palo Alto Calif.) and analyzed by the software MassLynx v3.3 (Waters, Milford Mass.).

Gel Electrophoresis and western blotting. Sodium dodecyl sulfate polyacrylamide gel electrophoresis (SDS-PAGE) analyses were performed using 4-12% gradient gels according to the method of Laemmli. In several experiments, proteins were transferred to polyvinylidene difluoride (PVDF) membranes according to the method described by Towbin et al. After transfer to nitrocellulose, factor V heavy and light chain(s) were detected using the appropriate monoclonal and polyclonal antibodies. Immunoreactive fragments were visualized with chemiluminescence.

### Results

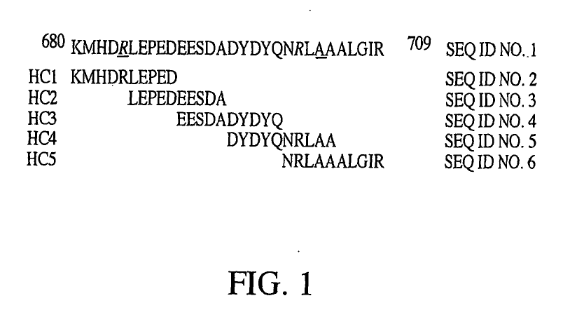

Inhibition of prothrombinase function by synthetic peptides from the carboxyl-terminal portion of factor Va heavy chain. There is increasing evidence that region 680-709 (SEQ ID NO. 1) of the heavy chain of factor Va is important for cofactor activity. This hirudin-like region containing functionally important tyrosine residues was proposed to provide an important site for the productive interaction with proexosite I of prothrombin. To explicitly identify the amino acid residues from this region that are important for cofactor activity, five overlapping peptides (ten amino acids each) spanning the entire region of interest (HC1-HC5, FIG. 1) (SEQ ID NO. 2-6, respectively) were synthesized. Each synthetic peptide, except the first and the last, has five amino acids in common with the preceding and the following peptide of the series.

Specifically, FIG. 1 illustrates peptides from amino acid region 680-709 (SEQ ID NO. 1) of factor Va. Overlapping peptides (10 residues each) from the COOH-terminal portion of the heavy chain of human factor Va are shown (amino acid region 680-709, HC1-HC5). The arginines are identified (bold and underlined). Lys⁶⁸⁰ and Arg⁷⁰⁹ are identified as the beginning and the end respectively of the sequence of interest.

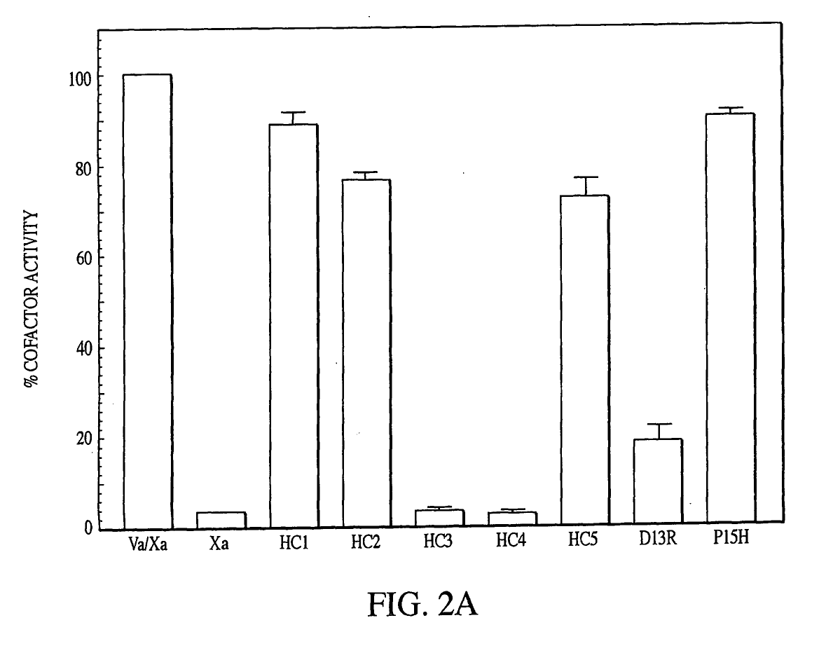

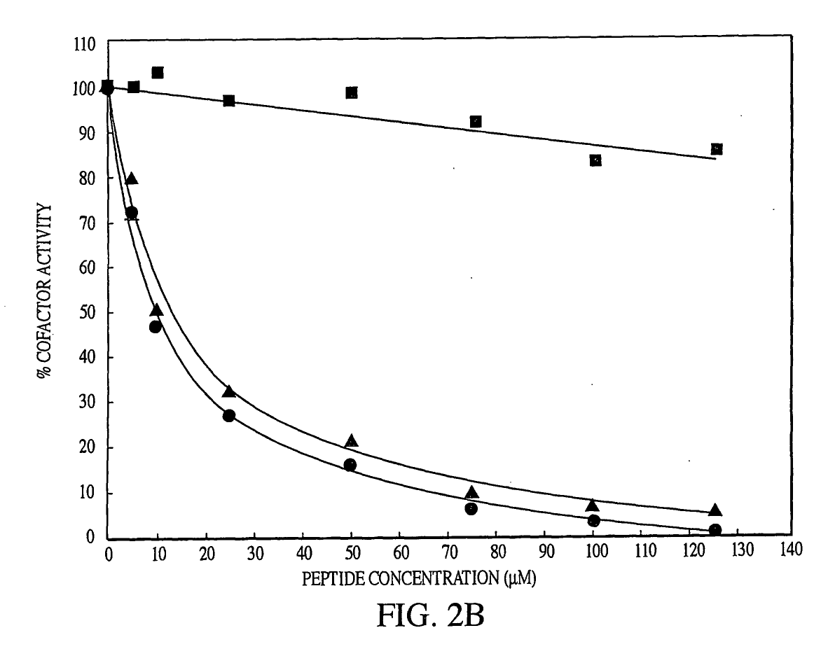

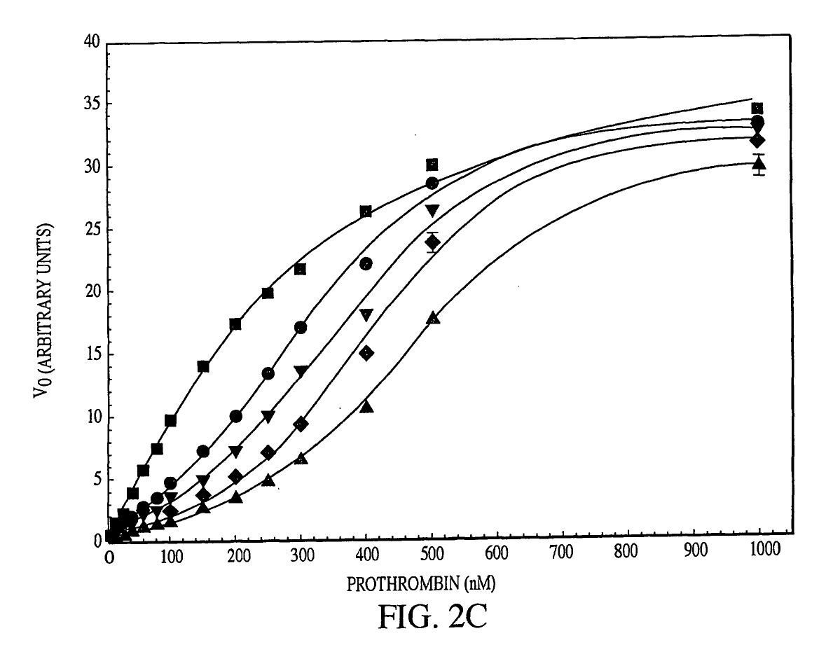

Under the conditions employed, two peptides, HC3 (SEQ ID NO. 4), and HC4 (SEQ ID NO. 5) (spanning amino acid regions 690-699 and 695-704 of factor Va heavy chain respectively) inhibited prothrombinase when used at 100 μM (FIG. 2A).

Specifically, peptides were incubated with factor Va as described herein at a fixed concentration (100 μM). The percent of factor Va cofactor activity was calculated by comparing the activity of prothrombinase in the presence of a given peptide to the activity of prothrombinase determined in a control reaction in the absence of peptide and in the presence of factor Xa. The amino acid sequence and identification of each peptide are given in FIG. 1. The data represent the average of the results found in three independent measurements. The control peptide represents a pentadecapeptide from the middle portion of factor Va heavy chain (P15H) (SEQ ID NO. 22). FIG. 2A also shows a positive control peptide, D13R (SEQ ID NO. 21), recently shown to interfere with prothrombin incorporation into prothrombinase and a negative control peptide, P15H, that has no effect on cofactor activity under the conditions employed. Control experiments also demonstrated that the peptides do not interfere with the capability of thrombin to interact with DAPA (not shown). All other peptides from region 680-709 had no significant effect on prothrombinase activity. Overall the data also show that amino acid sequences 680-689 and 700-709 which together represent approximately 67% of the entire amino acid sequence studied, do not appear to have any major effect on prothrombinase function under the conditions employed.

A titration of the inhibition of prothrombinase by peptides HC3 and HC4 is shown in FIG. 2B. Specifically, as shown in FIG. 2B, increasing concentrations of HC3 (filled triangles), HC4 (filled circles), and P15H (filled squares) were preincubated with factor Va and assayed for prothrombinase activity as described herein. HC3 represents amino acid sequence 690-699 of factor Va heavy chain; HC4 contains the sequence 695-704 of human factor Va heavy chain; P15H represents amino acid sequence 337-351 from the middle portion of human factor Va heavy chain. The data represent the average of the results found in three independent experiments. The concentration of each peptide given on the x axis represents its final concentration in the prothrombinase mixture. The data demonstrated that HC3 and HC4 inhibit prothrombinase activity with similar IC₅₀'s of 12 μM (FIG. 2B, filled triangles) and 10 μM (FIG. 2B, filled circles) respectively. In the presence of 100 μM HC3 and HC4 complete inhibition of prothrombinase function was observed (FIG. 2B). Thus, both peptides show similar inhibitory potential. A control pentadecapeptide from the middle portion of the heavy chain of factor Va (P15H) had no effect on prothrombinase function at similar concentrations under the conditions employed (FIG. 2B, filled squares).

HC3 and HC4 were also tested for their ability to interfere with the fluorescence anisotropy of a preformed complex composed of membrane-bound [OG₄₈₈]-EGR-hXa-human factor Va as described. No significant decrease in the anisotropy of [OG₄₈₈]-EGR-hXa was detected following incubation of the preformed complex with increasing concentrations of either HC3 or HC4 even in the presence of high concentrations of peptide (300 μM, not shown). The data demonstrate that peptides HC3 and HC4 do not interfere with the high affinity interaction between factor Va and factor Xa on the membrane surface. The peptides must thus impair another function of the cofactor within prothrombinase.

The mechanism of inhibition of prothrombinase by the hirudin-like peptides HC3 and HC4 was addressed by investigating the effect of the peptides on the kinetic parameters of the enzymatic complex (Kₘ and Vₘₐₓ). For the sake of simplicity only the results obtained with HC4 are shown (FIG. 2C). In FIG. 2C, the data are plotted as V₀ (initial velocity, in arbitrary units) as a function of increasing prothrombin concentration in the presence of increasing concentrations of HC4. The lines drawn represent the best fit through the points with an R² of 0.99. The concentrations of HC4 used in the experiments are as follows: control no peptide (filled squares), 0.5 μM peptide (filled circles), 1 μM peptide (filled inverted triangles), 1.5 μM peptide (filled diamonds), and 2 μM peptide (filled triangles). The data represent the average of the results found in three independent experiments. The apparent inhibition constant (Kᵢ) reported in the text is the value calculated from the formula: IC₅₀=Kᵢ(1+S₀/Kₘ), where Kₘ is the Michaelis-Menten constant of the reaction in the absence of inhibitor (0.4 μM), S₀ is the concentration of prothrombin used (350 nM), and IC₅₀ is the half maximal inhibition of prothrombinase by HC4 (FIG. 2B, 10 μM).

The data demonstrated that under the conditions employed and in the presence of increasing concentrations of peptide, all reactions tend towards the same asymptotic value which is the Vₘₐₓ of the reaction. The calculated values of the Vₘₐₓ of prothrombinase remained approximately unchanged (˜525±30 nM IIa/min), while the K_{0.5} of the enzymatic reactions increased. These results suggest a competitive type of inhibition. Examination of the data shown in FIG. 2C demonstrated a sigmoidal shape of the graphs, which becomes more pronounced as the concentration of HC4 was increased (from 0.5 μM, filled circles to 2 μM, filled triangles). These data represent a competitive inhibition mechanism where only free substrate (prothrombin) can produce thrombin in the presence of prothrombinase. According to this model, HC4 binds prothrombin in competition with the binding of prothrombin to prothrombinase (membrane-bound factor Va-factor Xa). The Kᵢ of prothrombinase inhibition by HC3 obtained from the value of the IC₅₀ derived from FIG. 2B using the value of the Kₘ (0.4 μM) determined in FIG. 2C (filled squares) was 6.3 μM, while the Kᵢ for prothrombinase inhibition by HC4 was determined to be 5.3 μM. These values represent the K_{D} of HC3 and HC4 for their interaction with prothrombin. Overall the data demonstrate that HC3 and HC4 do not interfere with the binding of factor Va to factor Xa but rather impair prothrombinase activity by inhibiting the direct interaction of the cofactor with prothrombin. An interference of the peptides with the membrane binding properties of the cofactor must be excluded since it has been demonstrated that a factor Va molecule lacking a portion or the entire acidic COOH-terminal peptide from the heavy chain binds to the lipid bilayer with similar affinity as the purified intact plasma cofactor.

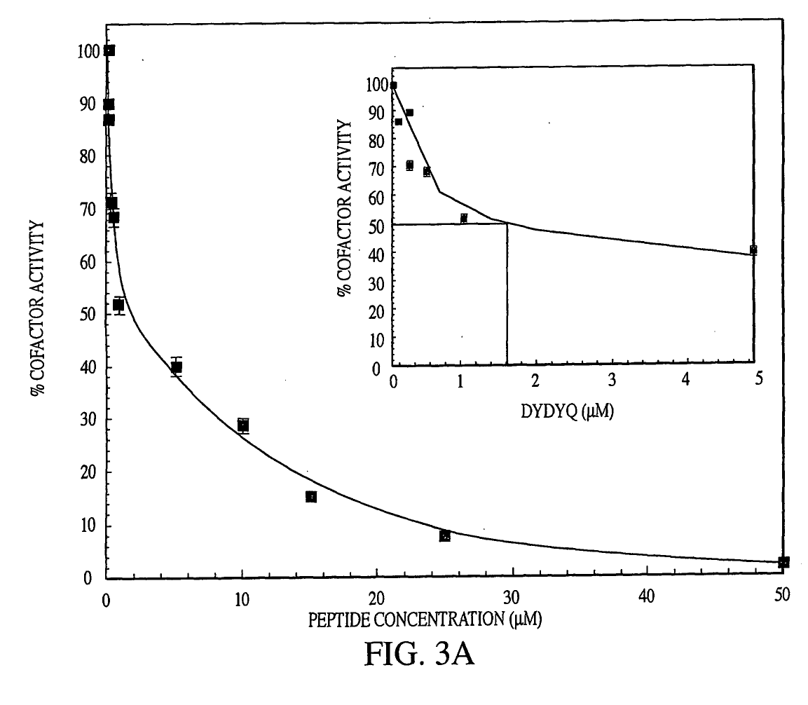

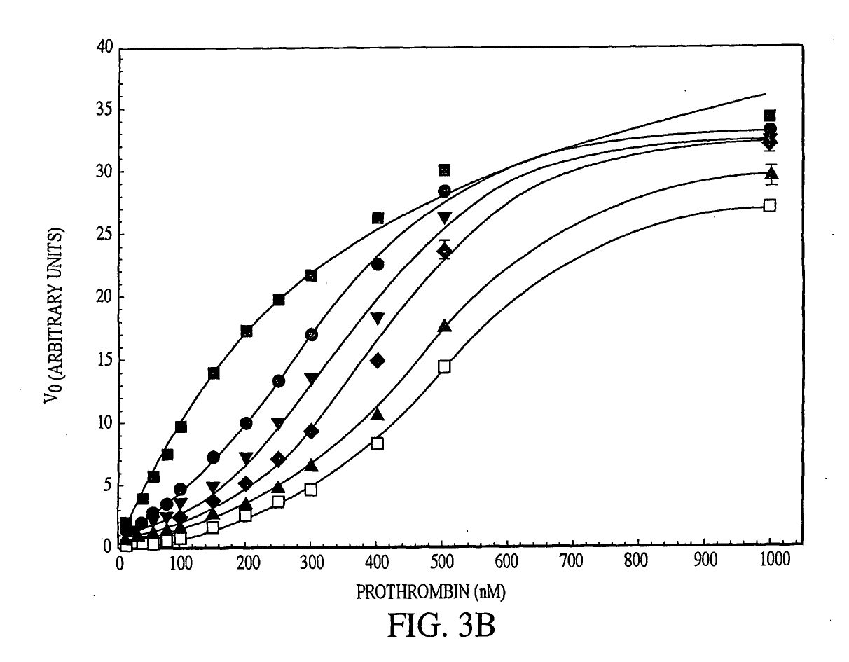

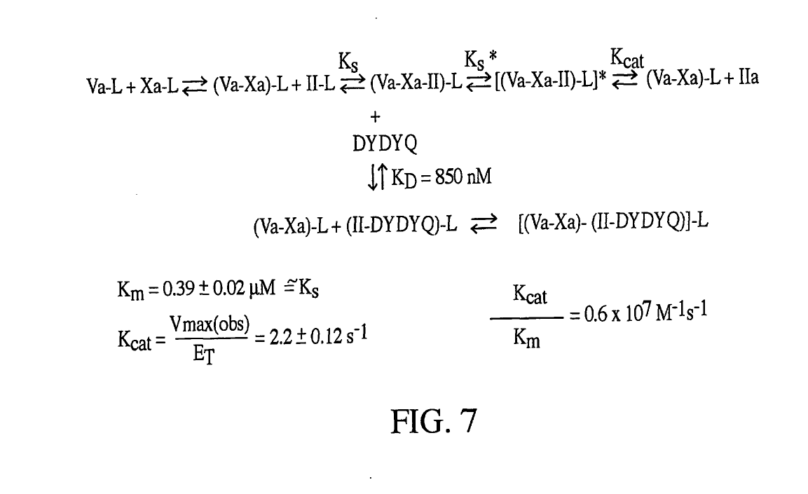

Function of the amino acid sequence common to HC3 and HC4. The common amino acid motif between HC3 and HC4 consists of amino acids residues Asp⁶⁹⁵-Tyr⁶⁹⁶-Asp⁶⁹⁷-Tyr⁶⁹⁸-Gln⁶⁹⁹ (DYDYQ) (SEQ ID NO. 11). A peptide with this sequence was found to be a potent inhibitor of prothrombinase function with an IC₅₀ of 1.6 μM (FIG. 3A). In FIG. 3A, increasing concentrations of DYDYQ (filled squares), were preincubated with factor Va and assayed for prothrombinase activity as described herein and in FIG. 2. The inset to FIG. 3A shows the progress of the reaction in the presence of 0-5 μM pentapeptide and allows for a more precise calculation of the IC₅₀ for prothrombinase inhibition which in turn is necessary for the calculation of the Kᵢ of the pentapeptide. The latter number represents K_{D} Of the inhibitor for its interaction with prothrombin (see FIG. 7). The data represent the average of the results found in three independent experiments. The concentration of peptide given on the x axis represents its final concentration in the prothrombinase mixture. Complete inhibition of prothrombinase by the pentapeptide occurred at 40 μM. Kinetic analyses of prothrombinase inhibition by DYDYQ similar to the analyses described for HC4 and shown in FIG. 2C revealed similar sigmoidal tracings in the presence of increasing concentrations of inhibitor (FIG. 3B).

In FIG. 3B, the data are plotted as V₀ (initial velocity, in arbitrary units) as a function of increasing prothrombin concentration in the presence of increasing concentrations of peptide DYDYQ. The lines drawn represent the best fit through the points with an R² of 0.99. The concentrations of DYDYQ used in the experiments are as follows: control no peptide (filled squares), 100 nM peptide (filled circles), 200 nM peptide (filled inverted triangles), 300 nM peptide (filled diamonds), 400 nM peptide (filled triangles), and 500 nM peptide (open squares). The data represent the average of the results found in three independent experiments. The apparent inhibition constant (Kᵢ) reported in the text was calculated as described in the legend to FIG. 2C using an IC₅₀ of 1.6 μM (FIG. 3A). The kinetic constants calculated from the data presented in this graph were used to calculate the k_{cat} reported in FIG. 7 assuming a final concentration of 4 nM prothrombinase. The sigmoidal nature of the curves and the K_{0.5} increased with increasing inhibitor concentration (100 nM (filled circles) to 500 nM (open squares)). Under the conditions employed the Kᵢ for inhibition of prothrombinase by the pentapeptide, which is the K_{D} for its interaction with prothrombin was determined to be 850 nM. The data suggest that the hirudin-like amino acid motif Tyr⁶⁹⁵-Gln⁶⁹⁹ from the COOH-terminal portion of the heavy chain of factor Va, represents a binding site for prothrombin within prothrombinase. The results verify the data found with HC4 and are consistent with the kinetic model of inhibition of prothrombinase shown in FIG. 7.

Specifically, FIG. 7 illustrates a kinetic model for inhibition of prothrombinase. Activation of prothrombin by prothrombinase is a multi step pathway. The initial bimolecular interaction responsible for enzyme formation on the membrane surface (L), is mediated by exosites from factor Va and factor Xa optimally exposed upon their interaction with the lipid surface. This complex interacts with membrane bound prothrombin (Kₛ) followed by docking of the scissile bonds in the active site of prothrombinase (Kₛ*). The last step of the reaction results in thrombin formation. The inhibitor (DYDYQ) interacts with prothrombin (K_{D}˜0.85 μM) in competition with the binding of prothrombin to prothrombinase (Kₛ˜0.4 μM). According to this model the true inhibitor of the enzymatic reaction is the prothrombin-DYDYQ complex which competes with free prothrombin for binding to prothrombinase and thrombin formation.

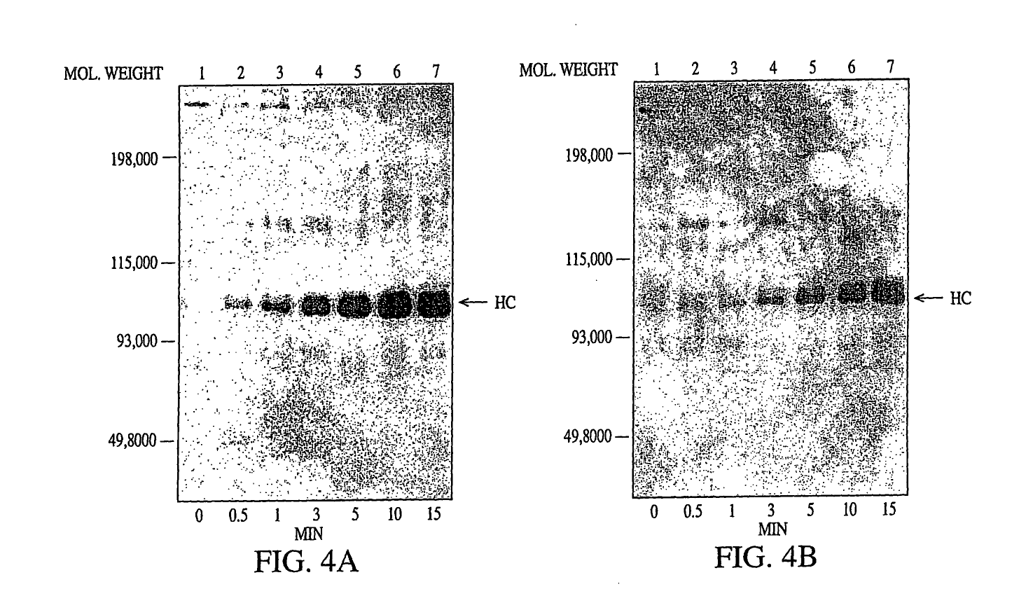

Since peptide DYDYQ is composed of acidic amino acids and most likely interacts with the positively charged amino acids from ABE-I of prothrombin as previously suggested, it is possible that the peptide also binds to thrombin and inhibits activation of factor V. Thus preincubated thrombin with 100 μM pentapeptide was tested as to the capacity of the mixture to activate single chain plasma factor V. Specifically, FIG. 4A shows activation of factor V by thrombin alone. Factor V (250 nM) was incubated with thrombin (1 nM). And FIG. 4B illustrates thrombin (1 nM) was preincubated with 100 μM peptide DYDYQ and the mixture was added to factor V (250 nM). At selected time intervals, aliquots of the mixtures were removed, mixed with 2% SDS, heated for 5 min at 90° C. and analyzed on a 4-12% SDS-PAGE followed by immunoblotting. Fragments were identified following staining with monoclonal antibody αHFV#17 that recognizes an epitope between amino acid residues 307-506 of factor V and chemiluminescence as described. Lane 1 in both panels depicts aliquots of the mixture withdrawn from the reaction before the addition of thrombin or thrombin/peptide mixture while lanes 2-7 show aliquots of the reaction mixture withdrawn at 30 sec, 1 min, 3 min, 5 min, 10 min, 15 min following the addition of thrombin alone or of thrombin/peptide mixture. The time of incubation is shown at the bottom of the figure. Position of the molecular weight markers is indicated at left.

Under the conditions employed the pentapeptide is a potent inhibitor of factor V activation by thrombin (FIG. 4, lanes 2-7) because it impairs cleavage at Arg⁷⁰⁹ (FIG. 4B) which is the first required step during the sequential activation of factor V. It is noteworthy that a delay in cleavage at Arg⁷⁰⁹ and generation of the heavy chain of the cofactor by the pentapeptide was observed with highly purified, single chain factor V only. When using partially activated preparations of factor V, no delay in the generation of the heavy chain in the presence of the pentapeptide was observed. In contrast, using these latter preparations a slower disappearance of the single chain factor V molecule was apparent (not shown). These data suggest that the pentapeptide impairs cleavage at Arg⁷⁰⁹ on the intact procofactor only. Overall the data depicted in FIGS. 3 and 4 together with the data shown in FIG. 2 demonstrate that the acidic amino acid stretch Asp⁶⁹⁵-Gln⁶⁹⁹ located at the carboxyl-terminal part of the factor Va heavy chain appears to have a dual function; it provides an exosite for prothrombin docking within prothrombinase and it also serves as an interactive site for thrombin necessary for cleavage at Arg⁷⁰⁹ and activation of the procofactor.

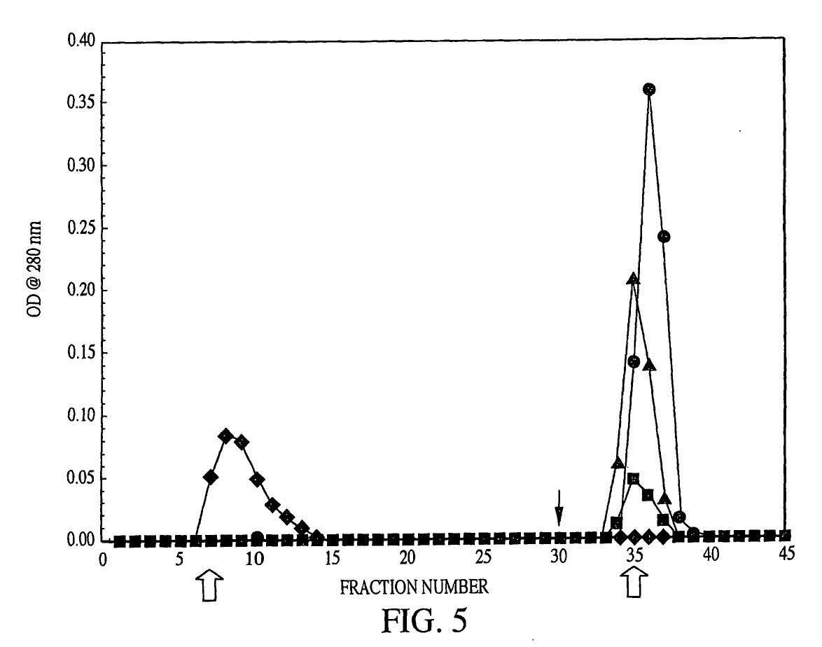

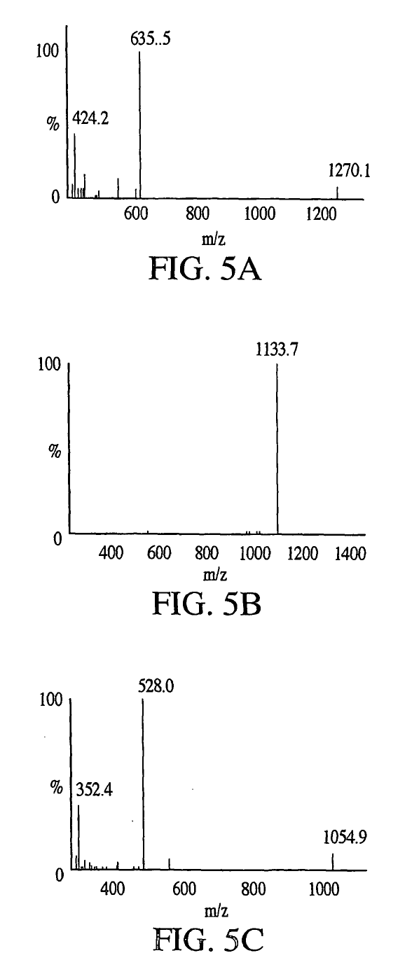

Direct interaction of hirudin-like peptides from factor Va heavy chain with thrombin-agarose. To ascertain that the peptides of interest are inhibitory of prothrombinase activity and contain a binding site for thrombin, the interaction of all peptides with active-site-immobilized thrombin was studied in chromatographic experiments and is shown in FIG. 5. In FIG. 5, small scale chromatography of hirudin-like peptides was performed on a 2.5 ml thrombin-agarose. Each run represents 400 μg of peptide. This amount of peptide was determined to saturate the specific sites of thrombin of the column used (2.5 ml). Elution was performed with 2M NaCl and was started at the point indicated by the arrow (fraction #30). The presence of the peptides in the corresponding fractions was monitored by absorbance at 280 nm (shown on the y axis) and by LC/MS (inset). Results show HC3 (filled squares), HC4 (filled triangles), DYDYQ (filled circles), and P15H (filled diamonds) monitored by the absorbance at 280 nm. In FIG. 5, the control peptide, P15H, did not interact with thrombin as demonstrated by its elution in the void volume of the thrombin-agarose column (FIG. 5, filled diamonds). All other peptides containing the hirudin-like motif DYDYQ were eluted from the column with high-salt buffer (FIG. 5; HC3 filled squares, HC4 filled triangles, and DYDYQ, filled circles). Two other peptides that could not be identified by the absorbance at 280 nm since they do not contain aromatic amino acids, but containing acidic amino acids and having the potential to interact with ABE-I or ABE-II of thrombin (HC1 and HC2, FIG. 1) were present in the flow-through of the thrombin-agarose column and detected by LC/MS (see FIGS. 5A and 5B). Specifically, these peptides were treated similarly to all other peptides and similar fractions were collected. Aliquots from fractions #7 and #35 from each separate experiment were submitted to LC/MS analysis as described herein (depicted by the hatched arrows at the bottom of the chromatogram). The results from tube #7 from three chromatograms are depicted. The data are presented as % intensity of the signal as a function of the mass of the peptide divided by the charge (m/z). FIG. 5A, HC1 (M_{r calculated} 1268); FIG. 5B, HC2 (M_{r calculated} 1132); FIG. 5C, HC5 (M_{r calculated} 1053). The spectrum of HC1 has two major peaks: at 635.5 and at 424, and one minor peak at 1270 (1268+1). The peak at 635.5 represents the peptide (mass/charge) with two positive charges ([M+2H]⁺) i.e. [(1268+2=1270)]/2=635 whereas, the peak at 424 represents HC1 with three positive charges ([M+3H]²⁺) i.e. [(1268+3=1271)]/3=424. Inset B, shows HC2 with one positive charge (1132+1=1133). HC5 spectrum has two major peaks: one at 528 and one at 352. The peak at 528 represents the peptide with two positive charges ([M+2H]⁺) i.e. [(1053+2=1055)]/2=527.5 while, the other peak represents HC5 with three positive charges ([M+3H]²⁺) i.e. [(1053+3=1056)]/3=352. The minor peak at 1054 represents the peptide with one charge. It is noteworthy, that in gas phase the number of protons attached to each peptide depends on the number (quantity) of basic residues contained in each peptide. HC1 contains one lysine and one arginine, HC2 does not contain any basic residues, while HC5 has two arginines (see FIG. 1). Thus, HC1 and HC5 are more likely to bind two or three protons than HC2. Consequently the species with one proton is the major species observed in the mass spectrum of HC2 (FIG. 5B). These data demonstrate that not any randomly selected acidic amino acid sequence can interfere with the binding of factor Va to the anionic binding exosite(s) of thrombin and strongly suggest that amino acid sequence Asp⁸⁹⁵-Gln⁶⁹⁹ contained in three of the seven peptides tested is a specific amino acid motif which represents a binding site for thrombin.

Expression and activation of recombinant human factor V molecules. In view of all these findings recombinant technology was utilized to assess the contribution of four out of the five amino acid residues identified above on both factor V activation and factor Va cofactor function. Two charge reversal and two conservative mutations were introduced into the 695-698 sequence of factor V. A quadruple mutant factor V molecule was prepared with the mutations Asp⁶⁹⁵→Lys, Tyr⁶⁹⁶→Phe, Asp⁶⁹⁷→Lys, and Tyr⁶⁹⁸→Phe (factor V^{2K2F}). Recombinant wild type factor V and factor V^{2K2F} were expressed in COS-7L cells, and their concentrations were determined using the ELISA recently developed.

The recombinant molecules were first screened for clotting activity and the results are shown in Table 1, as set forth below.

TABLE 1
Clotting Activity of Various Recombinant Factor V Species¹

| | Clotting time | Activity | Specific activity |
|---|---|---|---|
| Factor V Species² | (sec) | (U/ml) | (U/mg) |
| Media from mock | 67.6 ± 1.1 | — | — |
| transfected cells | | | |
| Wild type FV | 32.1 ± 1.2 | 0.12 | 145 |
| Wild type FV_{IIa} | 20.5 ± 0.6 | 0.44 | 497 |
| Wild type FV_{RVV} | 18.8 ± 0.5 | 0.47 | 570 |
| Factor V^{2K2F} | 66.7 ± 1.3 | 0.0046 | 5.6 |
| Factor Va_{IIa}^{2K2F} | 68.7 ± 4.8 | 0.0042 | 5.1 |
| Factor Va_{RVV}^{2K2F} | 73.1 ± 4 | 0.002 | 2.4 |

¹Wild type and mutant factor V molecules were assayed for clotting activity as described herein.
²All factor V species were assayed for clotting activity at 2.5 nM as described herein.

Wild type factor V had a specific activity of 145 U/mg. Activation of the wild type molecule by thrombin or RVV-V activator resulted in cofactors with similar clotting activities (497 U/mg and 570 U/mg respectively, Table 1). The quadruple mutant (factor V^{2K2F}) was unable to promote clotting under the conditions employed. Factor V^{2K2F} was also unable to promote clotting following activation by thrombin (factor Va_{IIa}^{2K2F}) and/or RVV-V activator (factor Va_{RVV}^{2K2F}). The two cofactor molecules had an activity analogous to the activity of the media collected from mock-transfected cells (<0.2% of the clotting activity of the wild type molecules activated under similar conditions Table 1). These data demonstrate that once activated factor Va_{IIa}^{2K2F} and factor Va_{RVV}^{2K2F} are deficient in their clotting activity.

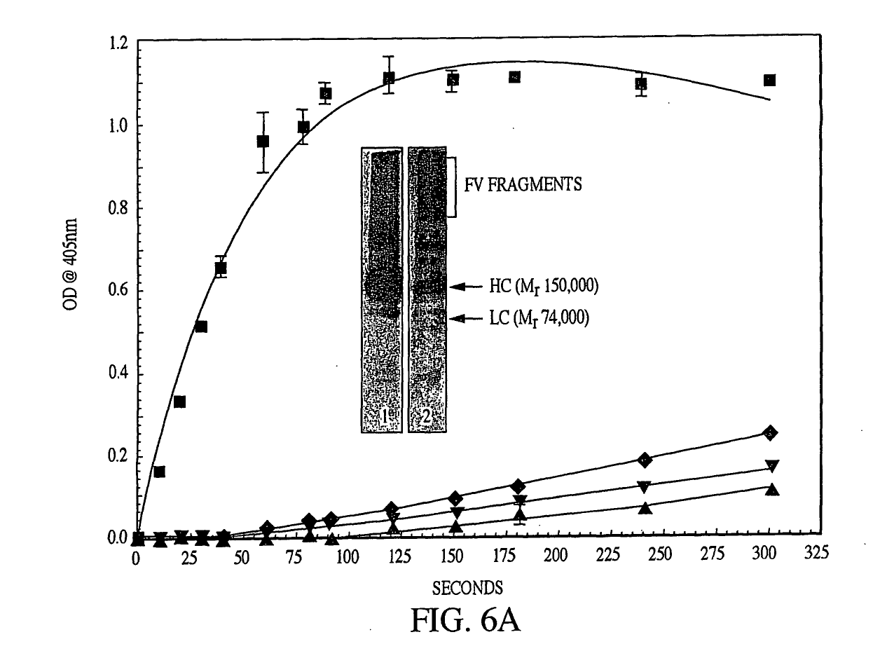

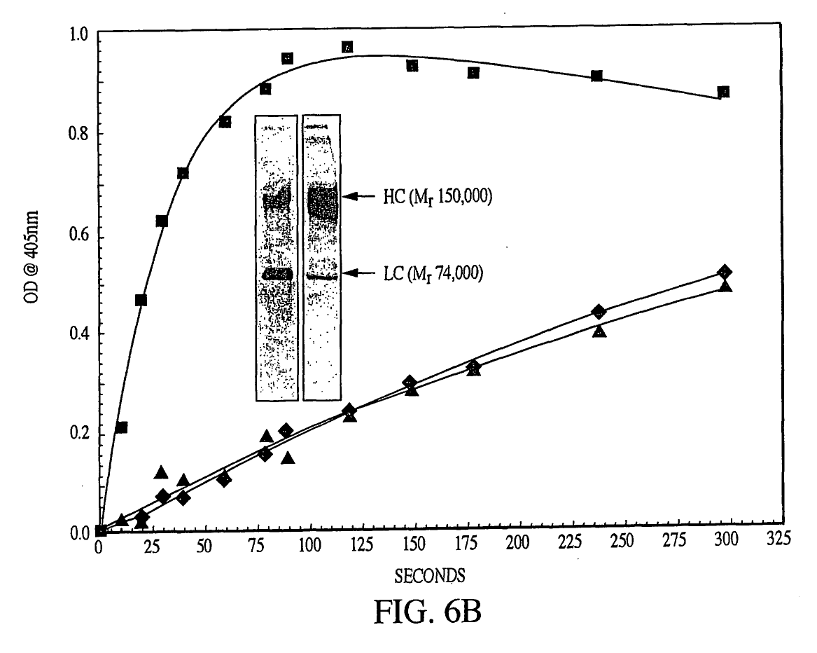

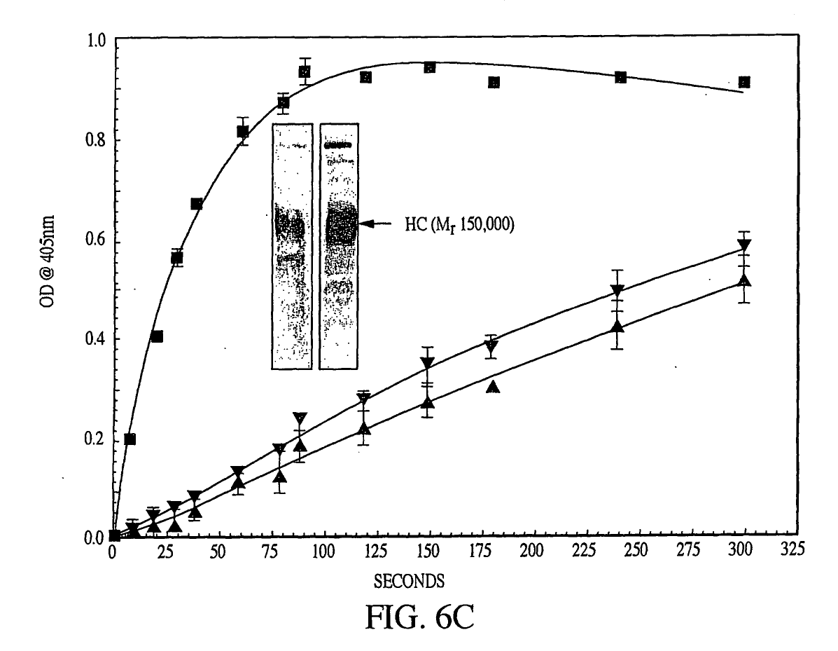

The ability of the recombinant molecules to be assembled into prothrombinase using an assay employing purified reagents and a chromogenic substrate that measures thrombin generation was also investigated. Since the assay is conducted with limiting factor Va concentrations (0.5 nM), any intrinsic deficiency in the activity of the complex reflects the inability of the recombinant mutant molecule to act as a cofactor in prothrombinase. FIG. 6A shows the results obtained following incubation of the procofactors with thrombin. Specifically, in FIG. 6A, the factor V species were activated by thrombin (10 nM, 15 min at 37° C.). The subunit composition of the thrombin activated species was also analyzed on a 4-12% SDS-PAGE followed by transfer to PVDF and immunostaining with monoclonal antibodies αHFV_{HC}#17 and αHFV_{LC}#9 (inset). Lane 1, wild type factor V following the incubation with thrombin; lane 2 factor V^{2K2F} following incubation with thrombin under similar experimental conditions. The cofactor activities of various factor Va species are depicted as follows: filled squares, wild type recombinant factor Va (0.5 nM); filled triangles, factor V^{2K2F}/Va^{2K2F} solution (0.5 nM); filled inverse triangles, factor V^{2K2F}/Va^{2K2F} solution (5 nM); filled diamonds, factor V^{2K2F}/Va^{2K2F} solution (25 nM). The data represent the average of the results found in three independent experiments. HC and LC represent the heavy (Mᵣ 105,000) and light (Mᵣ 74,000) chains of the cofactor respectively. The data demonstrate that the wild type recombinant factor Va molecule displays normal cofactor activity (˜1800 mOD/min, filled squares) under the conditions employed and is composed of heavy and light chains (FIG. 6A, inset lane 1). Under similar experimental conditions, factor V^{2K2F} activation by thrombin was impaired. The inset in FIG. 6A, lane 2 shows that, while there was some generation of heavy and light chains of mutant factor Va following a 15 min incubation with thrombin, considerable amounts of high molecular weight material remained on top of the gel. Furthermore, the factor V^{2K2F}/Va_{IIa}^{2K2F} mixture had no cofactor activity when compared with the wild type factor Va molecule (≦20 mOD/min); its cofactor activity was similar to the activity of factor Xa alone. The activity of the factor V^{2K2F}/Va_{IIa}^{2K2F} solution within prothrombinase remained essentially the same even when 10- and 50-times more total protein was used (FIG. 6A, 5 nM filled inverse triangles, 25 nM filled diamonds). The slow increase in the activity of the factor V^{2K2F}/Va_{IIa}^{2K2F} solution after two minutes of incubation in the reaction mixture is consequence of the slow activation of the molecule during the course of the assay by factor Xa and confirms that the mutant procofactor cannot be efficiently activated by thrombin. Slow activation of factor V during the course of the prothrombinase assay by factor Xa and/or thrombin generated in situ was previously observed when studying prothrombinase activity in the presence of unactivated factor V.

In FIG. 6B, the recombinant factor V species were activated with RVV-V activator (6 nM, 2 hr at 37° C.). The subunit composition of the RVV-activated species was also analyzed on a 4-12% SDS-PAGE after reduction with 2% β-mercaptoethanol followed by transfer to PVDF and immunostaining with monoclonal antibodies αHFV_{HC}#17 and αHFV_{LC}#9 (inset). Lane 1, wild type factor V following incubation with RVV; lane 2 factor V^{2K2F} following incubation with RVV. The cofactor activities of various recombinant factor Va species are depicted as follows: filled squares, wild type recombinant factor Va (0.5 nM); filed triangles, factor Va_{RVV}^{2K2F} (0.5 nM); filled diamonds, factor Va_{RVV}^{2K2F} (25 nM). The data represent the average of the results found in two independent experiments. HC and LC represent the heavy (Mᵣ 150,000) and light (Mᵣ 74,000) chains of the RVV-activated cofactors respectively. In FIG. 6C, the factor V species were activated with factor Xa (5 nM, 20 min in the presence of 20 μM PCPS vesicles at 37° C.). The amount of factor Xa brought in the assay from the activation mixtures was accounted for in the calculation of the final concentration of factor Xa (5 nM final concentration). The subunit composition of the factor Xa-activated species was also analyzed on a 4-12% SDS-PAGE after reduction with 2% β-mercaptoethanol followed by transfer to PVDF and immunostaining with monoclonal antibodies αHFV_{HC}#17 and αHFV_{LC}#9 (inset). The cofactor activities of various factor Va species are depicted as follows: filled squares, wild type recombinant factor Va (0.5 nM); filled triangles, factor Va_{Xa}^{2K2F} (0.5 nM); filled inverse triangles, factor Va_{Xa}^{2K2F} (5 nM). The data represent the average of the results found in three independent experiments. HC represents the heavy chain (Mᵣ 150,000) of the factor Xa-activated cofactors. Upon prolonged exposure of the immunoblots the Mᵣ 105,000 heavy chain of the cofactor was also apparent. In all insets the mutant molecules were consistently overloaded on the gels in order to identify any abnormal fragments and/or migration.

The results shown in FIGS. 6B and 6C demonstrate that activation of the wild type molecule by RVV-V activator (FIG. 6B, filled squares) or by factor Xa (FIG. 6C, filled squares) results in cofactors with similar activities as the thrombin-activated molecule (FIG. 6A, filled squares). However, under similar experimental conditions factor Va_{RVV}^{2K2F} and factor Va_{Xa}^{2K2F} have similar but still impaired cofactor activities within prothrombinase (FIG. 6B, filled triangles, and FIG. 6C, filled triangles). Prothrombinase activity does not increase with increasing cofactor concentration (FIG. 6B, filled diamonds, and FIG. 6C, filled inverse triangles). While the activity of these cofactors within prothrombinase is approximately six times higher than the cofactor activity obtained with the thrombin activated solution of factor V^{2K2F}/Va_{IIa}^{2K2F}, factors Va_{RVV}^{2K2F} and Va_{Xa}^{2K2F} have approximately 22 times less cofactor activity than the wild type molecule activated under similar experimental conditions. It is noteworthy that prolonged incubation of factor V^{2K2F} with thrombin resulted in a cofactor molecule (factor Va_{IIa}^{2K2F}) with similar activity as factors Va_{RVV}^{2K2F} and Va_{Xa}^{2K2F} (not shown). Altogether the data shown in FIG. 6 demonstrate that: 1) factor V^{2K2F} is impaired in its activation by thrombin; and 2) the activated mutant cofactor (factor Va_{RVV}^{2K2F} and factor Va_{Xa}^{2K2F}) are impaired in their intrinsic function within prothrombinase most likely because of impaired interaction with prothrombin.

Finally, it is important to note that several studies utilizing high resolution x-ray crystal structures of coagulation and fibrinolytic enzyme complexes have suggested that while the active-site geometry of the enzyme component from several procoagulant and fibrinolytic complexes does not appear to be altered upon incorporation of the corresponding protein cofactor into the complex, docking of a hidden cleavage site of the substrate into the active-site cleft of the enzyme following binding of the cofactor molecule to the substrate appeared to promote enzymatic specificity and optimum catalysis by providing an extended binding surface for the substrate. The findings presented herein are in complete agreement with these previous results, and provide for the first time a functional demonstration for cofactor directed catalysis of an enzymatic complex.

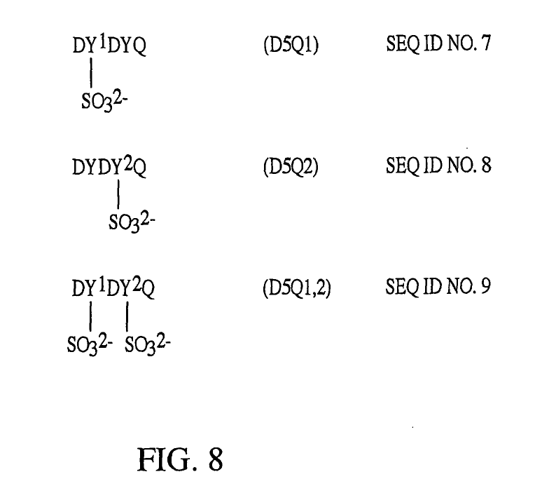

As previously noted, another significant aspect of the present discovery relates to certain sulfonated peptides. Essentially, these peptides correspond to several of the previously described peptides of interest however, also include one or more sulfonate groups within the amino acid sequence of interest. Examples of these peptides include, but are not limited to, the peptides illustrated in FIG. 8 and designated as (D5Q1) (SEQ ID NO. 7), (D5Q2) (SEQ ID NO. 8), and (D5Q1,2) (SEQ ID NO. 9). It will be understood that a shorthand designation for peptides is made by a reference to the first amino acid, then a number of the total amino acids in the peptide, and then, a reference to the last amino acid of the peptide. Thus, for the peptide DYDYQ, the shorthand designation is “D5Q”. The numeric suffix to the shorthand designation such as shown in FIG. 8, i.e. “D5Q1” refers to which of the Y amino acids is sulfonated. This shorthand designation format is periodically utilized in several of the accompanying patent figures. It will be appreciated that the designation D5Q1 is equivalent to DY(—SO₃)DYQ. Also, another equivalent designation to D5Q1 is “DYDYQ-1” as periodically noted on several of the patent figures. Other equivalent designations for D5Q2, D5Q1, 2 . . . etc. will be understood.

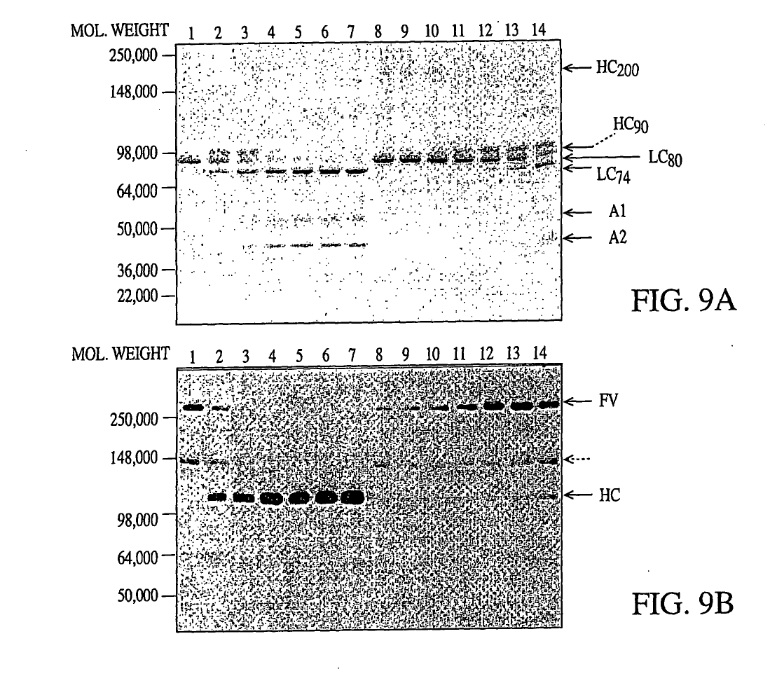

FIGS. 9A and 9B demonstrate the inhibition of activation of factor VIII and factor V respectively. Specifically, FIG. 9A illustrates the inhibition of activation of factor VIII by thrombin, the inhibition resulting from the double sulfonated peptide DYDYQ (1, 2) (SEQ ID NO. 9) binding to thrombin. FIG. 9B illustrates the inhibition of activation of factor V by thrombin, the inhibition resulting from the double sulfonated peptide DYDYQ (1, 2) (SEQ ID NO. 9) binding to thrombin. FIGS. 9A and 9B result from analysis by SDS-PAGE as previously described with regard to FIG. 4(A and B). These figures reveal that DYDYQ (1, 2) is a potent inhibitor of factors VIII (FIG. 9A) and factor V (FIG. 9B) because this peptide impairs cleavage of the respective factor which is a required step during the sequential activation of the respective factor. The various fragments of each factor are noted on the right hand side of each figure.

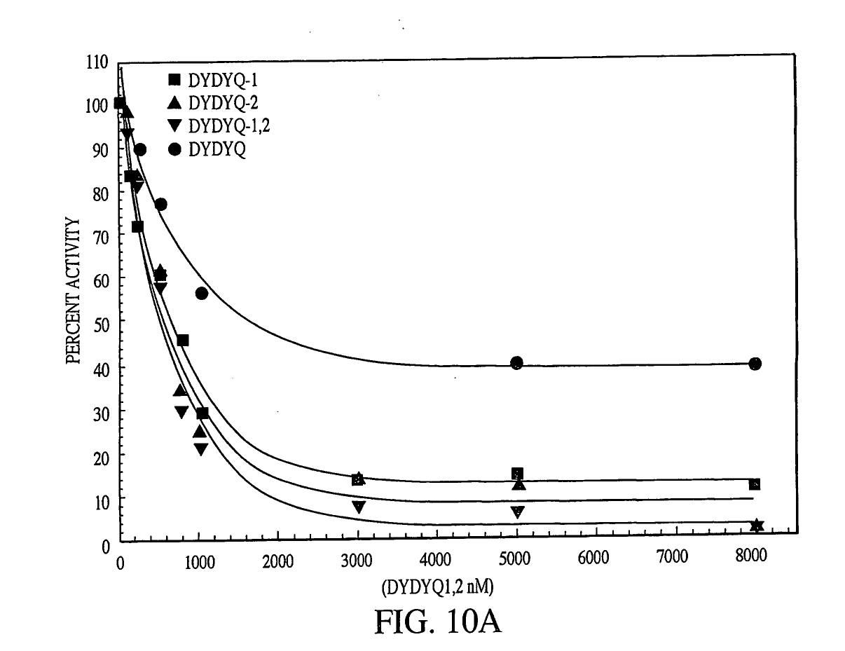

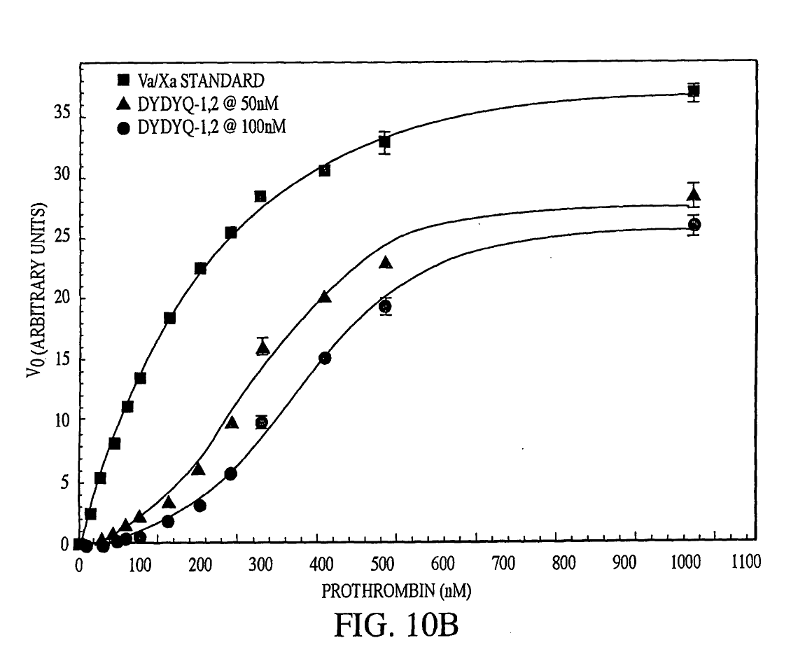

FIG. 10A illustrates the inhibition of prothrombinase by the sulfonated peptides of interest, namely DYDYQ-1 (SEQ ID NO. 7); DYDYQ-2 (SEQ ID NO. 8); and DYDYQ-1,2 (SEQ ID NO. 9) as compared to another non-sulfonated peptide of interest DYDYQ (SEQ ID NO. 11). Specifically, as shown in FIG. 10A, increasing concentrations of all these peptides, i.e.—DYDYQ-1 (filled squares), DYDYQ-2 (filled triangles), DYDYQ-1,2 (filled inverted triangles), and DYDYQ (filled circles), resulted in a decrease in the activity of prothrombinase, as described herein. The concentration of each peptide on the X axis is noted in nanomoles, and up to 8000 nM. Although the extent of inhibition of the peptide DYDYQ is attractive, the significantly greater inhibitory function of the sulfonated peptides is surprising. That is, the peptides DYDYQ-1, DYDYQ-2, and DYDYQ-1,2 exhibited significant inhibitory effects upon prothrombinase activity. It will be noted that the sulfonated peptide exhibiting the greatest inhibitory effect of this trial, is DYDYQ-1,2.

FIG. 10B illustrates the effect of increasing concentration of the peptide DYDYQ-1,2 (SEQ ID NO. 9) upon the reaction kinetics of prothrombinase. Specifically, this figure reveals the kinetics of prothrombinase inhibition in the presence of the double sulfonated peptide DYDYQ-1,2. In FIG. 10B, the data are plotted as Vₒ (initial velocity, in arbitrary units) as a function of increasing prothrombin concentration in the presence of increasing concentration of DYDYQ-1,2. The concentrations of DYDYQ-1,2 are 50 nM (filled triangles) and 100 nM (filled circles). A control of factors Va and Xa is shown (filled squares). As previously explained with regard to FIG. 2C, the sigmoidal shape of the curves for both concentrations of DYDYQ-1,2 indicate a competitive inhibition mechanism in which DYDYQ-1,2 binds prothrombin in competition with the binding of prothrombin to prothrombinase.

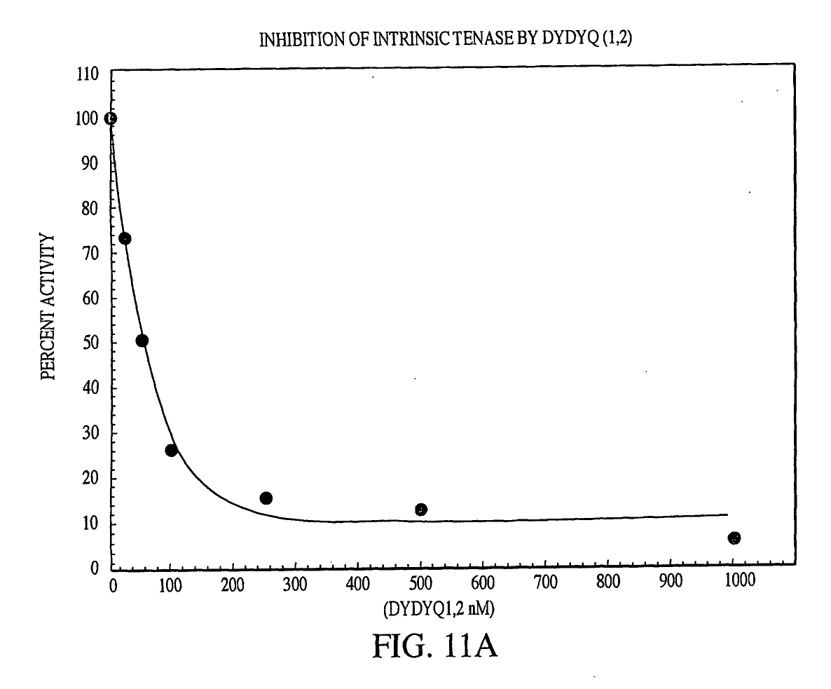

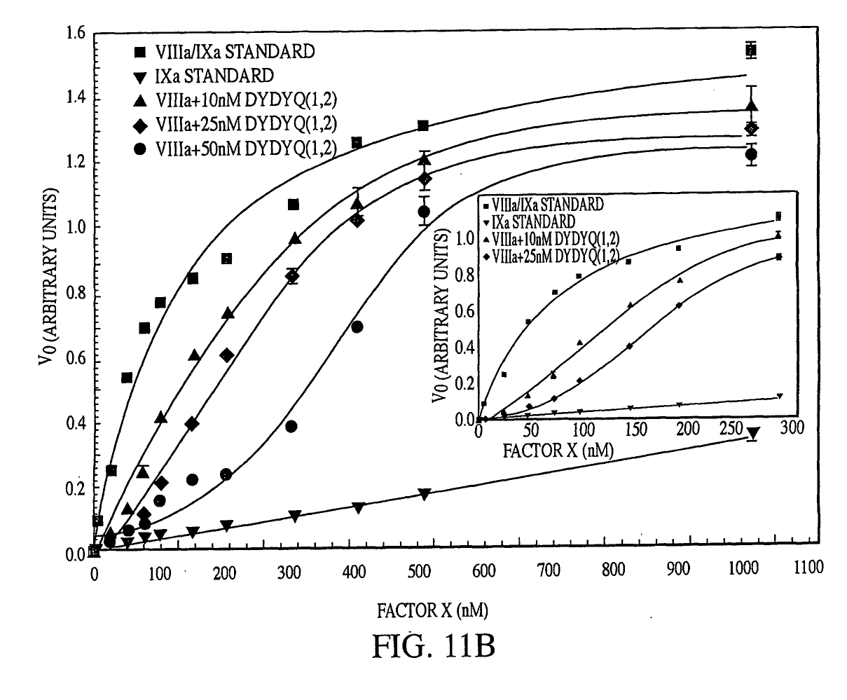

FIG. 11A illustrates inhibition of intrinsic tenase by the double sulfonated peptide DYDYQ (1, 2) (SEQ ID NO. 9). It is remarkable that such a dramatic reduction in prothrombinase activity is realized at such relatively low concentrations of DYDYQ (1, 2). As is shown in FIG. 11A, as the concentration of DYDYQ (1, 2) is increased, the reduction in activity is dramatic.

FIG. 11B and its insert panel illustrate the effect of increasing concentration of the peptide DYDYQ (1, 2) (SEQ ID NO. 9) upon the reaction kinetics of intrinsic tenase. Specifically, this figure illustrates the kinetics of inhibition of intrinsic tenase in the presence of the double sulfonated peptide DYDYQ (1, 2). In FIG. 11B, the data are plotted as Vₒ (initial velocity in arbitrary units) as a function of increasing factor X concentration in the presence of increasing concentration of DYDYQ (1, 2). The concentrations of DYDYDQ (1, 2), in addition with factor VIIIa, are 10 nM (filled triangles), 25 nM (filled diamonds), and 50 nM (filled circles). Control curves of factors VIIIa and IXa (filled squares) and factor IXa (inverted triangles) are also noted. The insert panel of FIG. 11B is presented for greater clarity and details the region of factor X concentration up to 300 nM, and without the curve of 50 nM of DYDYQ (1, 2).

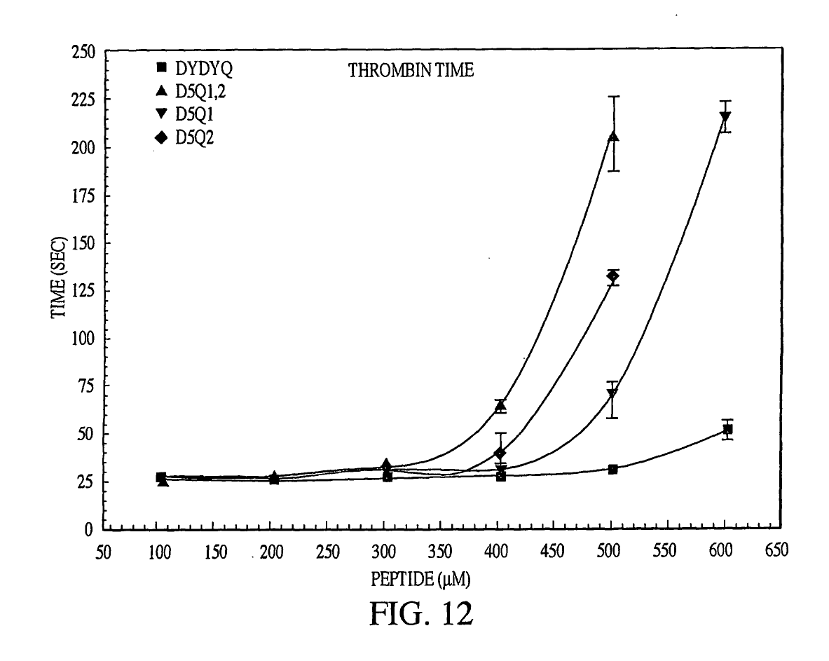

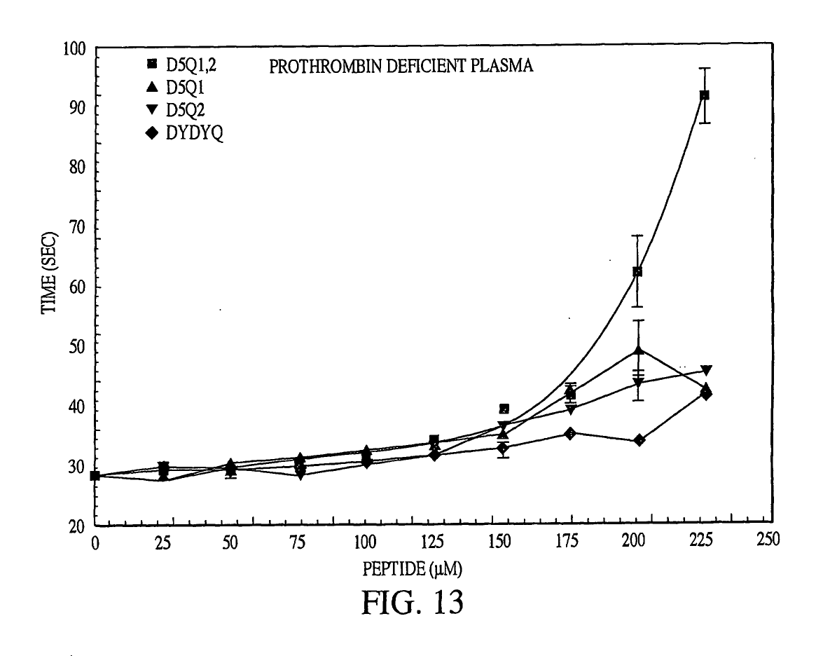

FIG. 12 is a graph illustrating clotting time as a function of concentration of various peptides of interest as follows: DYDYQ (SEQ ID NO. 11) (filled squares); DYDYQ (1, 2) (or as designated in shorthand form, D5Q1,2) (SEQ ID NO. 9) (filled triangles); DYDYQ-1 (SEQ ID NO. 7) (filled inverted triangles); and DYDYQ-2 (SEQ ID NO. 8) (filled diamonds). At a concentration of, for example, 500 nM, all peptides exhibited relatively long clotting times, with the peptide DYDYQ (1, 2) exhibiting the longest clotting time, and thus, greatest inhibitory effect.

FIG. 13 illustrates the effect upon clotting time of prothrombin deficient plasma by various peptides of interest, as follows: DYDYQ (1, 2) (SEQ ID NO. 9) (filled squares); DYDYQ-1 (SEQ ID NO. 7) (filled triangles); DYDYQ-2 (SEQ ID NO. 8) (filled inverted triangles); and DYDYQ (SEQ ID NO. 11) (filled diamonds).

The present discovery has been described with reference to various exemplary embodiments and aspects thereof. Obviously, modifications and alterations will occur to others upon reading and understanding the preceding detailed description. It is intended that the exemplary embodiments be construed as including all such modifications and alterations insofar as they come within the scope of the appended claims or the equivalents thereof.

1. A peptide having a sequence of amino acids which is identical to a sequence of consecutive amino acids found within amino acids 695 to 698 (SEQ ID NO. 10) of the human blood clotting factor Va.

2. The peptide of claim 1 wherein the peptide exhibits an IC₅₀ of less than about 100 μM, the IC₅₀ being the amount of the peptide that inhibits 50% of the activity of human factor Va.

3. The peptide of claim 2 wherein the peptide exhibits an IC₅₀ of less than about 15 μM.

4. The peptide of claim 3 wherein the peptide exhibits an IC₅₀ of about 1.6 μM.

5. The peptide of claim 4 wherein the peptide exhibits an IC₅₀ of about 500 nM.

6. The peptide of claim 1 wherein the peptide comprises the amino acid sequence DYDY.

7. The peptide of claim 1 wherein the peptide comprises the amino acid sequence DYDYQ.

8. A pharmaceutical composition comprising the peptide of claim 1.

9. (canceled)

10. A peptide analogue that mimics the peptide of claim 1.

11-42. (canceled)

43. A pharmaceutical composition adapted for inhibiting thrombin generation, the composition comprising a peptide including an amino acid sequence DYDY (SEQ ID NO. 10).

44. The pharmaceutical composition of claim 43 further comprising a carrier.

45. The pharmaceutical composition of claim 43 wherein one of the Y amino acids of the amino acid sequence is sulfonated.

46. The pharmaceutical composition of claim 45 wherein the amino acid sequence of the peptide is DY(—SO₃)DY.

47. The pharmaceutical composition of claim 45 wherein the amino acid sequence of the peptide is DYDY(—SO₃).

48. The pharmaceutical composition of claim 43 wherein both of the Y amino acids of the amino acid sequence are sulfonated.

49. The pharmaceutical composition of claim 48 wherein the amino acid sequence of the peptide is DY(—SO₃)DY(—SO₃).

50. (canceled)

51. A pharmaceutical composition comprising a peptide analogue that mimics the peptide of the composition of claim 43.

52-111. (canceled)

112. A peptide consisting of a sequence of four amino acids which is identical to a sequence of consecutive amino acids found within amino acids 695 to 698 (SEQ ID NO. 10) of the human blood clotting factor Va.

113. The peptide of claim 112 wherein the peptide comprises the amino acid sequence DYDY.

114. A pharmaceutical composition comprising the peptide of claim 112.

115. A peptide analogue that mimics the peptide of claim 112.

116. A peptide consisting of a sequence of five amino acids which is identical to a sequence of consecutive amino acids found within amino acids 695 to 699 (SEQ ID NO. 11) of the human blood clotting factor Va.

117. The peptide of claim 116 wherein the peptide comprises the amino acid sequence DYDYQ.

118. A pharmaceutical composition comprising the peptide of claim 116.

119. A peptide analogue that mimics the peptide of claim 116.

120. A pharmaceutical composition adapted for inhibiting thrombin generation, the composition comprising a peptide consisting of an amino acid sequence DYDY (SEQ ID NO. 10).

121. The pharmaceutical composition of claim 120 further comprising a carrier.

122. The pharmaceutical composition of claim 120 wherein one of the Y amino acids of the amino acid sequence is sulfonated.

123. The pharmaceutical composition of claim 122 wherein the amino acid sequence of the peptide is DY(—SO₃)DY.

124. The pharmaceutical composition of claim 122 wherein the amino acid sequence of the peptide is DYDY(—SO₃).

125. The pharmaceutical composition of claim 120 wherein both of the Y amino acids of the amino acid sequence are sulfonated.

126. The pharmaceutical composition of claim 125 wherein the amino acid sequence of the peptide is DY(—SO₃)DY(—SO₃).

127. A pharmaceutical composition comprising a peptide analogue that mimics the peptide of the composition of claim 120.

128. A pharmaceutical composition adapted for inhibiting thrombin generation, the composition comprising a peptide consisting of an amino acid sequence DYDYQ (SEQ ID NO. 11).

129. The pharmaceutical composition of claim 128 further comprising a carrier.

130. The pharmaceutical composition of claim 128 wherein one of the Y amino acids of the amino acid sequence is sulfonated.

131. The pharmaceutical composition of claim 130 wherein the amino acid sequence of the peptide is DY(—SO₃)DYQ.

132. The pharmaceutical composition of claim 130 wherein the amino acid sequence of the peptide is DYDY(—SO₃)Q.

133. The pharmaceutical composition of claim 128 wherein both of the Y amino acids of the amino acid sequence are sulfonated.

134. The pharmaceutical composition of claim 133 wherein the amino acid sequence of the peptide is DY(—SO₃)DY(—SO₃)Q.

135. A pharmaceutical composition comprising a peptide analogue that mimics the peptide of the composition of claim 128.


SEQUENCE LISTING (25 sequences)

SEQ ID NO: 1 - protein, 30 aa, Homo sapiens
KMHDRLEPQDEESDADYDYQNRLAAALGIR

SEQ ID NO: 2 - protein, 10 aa, Homo sapiens
KMHDRLEPED

SEQ ID NO: 3 - protein, 10 aa, Homo sapiens
LEPEDEESDA

SEQ ID NO: 4 - protein, 10 aa, Homo sapiens
EESDADYDYQ

SEQ ID NO: 5 - protein, 10 aa, Homo sapiens
DYDYQNRLAA

SEQ ID NO: 6 - protein, 10 aa, Homo sapiens
NRLAAALGIR

SEQ ID NO: 7 - protein, 5 aa, Artificial Sequence
  chemically synthesized
DYDYQ

SEQ ID NO: 8 - protein, 5 aa, Artificial Sequence
  chemically synthesized, TYR AT SEQUENCE LOCATION 4 IS SULFONATED (-SO3-2)
DYDYQ

SEQ ID NO: 9 - protein, 5 aa, Artificial Sequence
  chemically synthesized, TYR AT SEQUENCE LOCATIONS 2 AND 4 ARE SULFONATED (-SO3-2)
DYDYQ

SEQ ID NO: 10 - protein, 4 aa, Homo sapiens
DYDY

SEQ ID NO: 11 - protein, 5 aa, Homo sapiens
DYDYQ

SEQ ID NO: 12 - protein, 4 aa, Artificial Sequence
  chemically synthesized, TYR AT SEQUENCE LOCATION 2 IS SULFONATED (-SO3-2)
DYDY

SEQ ID NO: 13 - protein, 4 aa, Artificial Sequence
  chemically synthesized, TYR AT SEQUENCE LOCATION 4 IS SULFONATED (-S03-2)
DYDY

SEQ ID NO: 14 - protein, 4 aa, Artificial Sequence
  chemically synthesized, TYR AT SEQUENCE LOCATIONS 2 AND 4 ARE SULFONATED (-SO3-2)
DYDY

SEQ ID NO: 15 - DNA, 23 bp, Homo sapiens
gagtgatgctaagtttgattacc

SEQ ID NO: 16 - DNA, 23 bp, Homo sapiens
ggtaatcaaacttagcatcactc

SEQ ID NO: 17 - DNA, 18 bp, Homo sapiens
catggagtgaccttctcg

SEQ ID NO: 18 - DNA, 15 bp, Homo sapiens
tcatccaggagaacc

SEQ ID NO: 19 - DNA, 28 bp, Homo sapiens
gctaagtttaagttccagaacagactgg

SEQ ID NO: 20 - DNA, 28 bp, Homo sapiens
ccagtctgttctggaacttaaacttagc

SEQ ID NO: 21 - protein, 13 aa, Artificial Sequence
  chemically synthesized
DYQNRLAAALGIR

SEQ ID NO: 22 - protein, 15 aa, Artificial Sequence
  chemically synthesized
PVIPANMDKKYRSQH

SEQ ID NO: 23 - protein, 42 aa, Homo sapiens
NLKKITREQRRHMKRWEYFIAAEEVIWDYAPVIPANMDKKYR

SEQ ID NO: 24 - protein, 9 aa, Homo sapiens
EYFIAAEEV

SEQ ID NO: 25 - protein, 5 aa, Homo sapiens
EYFIA